SEQUENCE LISTING (20 sequences)

SEQ ID NO: 1 - protein, 64 aa, Homo sapiens
GPYHPSECCFTYTTYKIPRQRIMDYYETNSQCSLPGIVIITKRGHSVCTNPSDKWVQDYIKDMK

SEQ ID NO: 2 - protein, 63 aa, Homo sapiens
HPSECCFTYTTYKIPRQRIMDYYETNSQCSKPGIVFITKRGHSVCTNPSDKWVQDYIKDMKEN

SEQ ID NO: 3 - protein, 93 aa, Homo sapiens
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 4 - protein, 74 aa, Homo sapiens
TKTESSSRGPYHPSECCFTYTTYKIPRQRIMDYYETNSQCSKPGIVFITKRGHSVCTNPSDKWVQDYIKDMKEN

SEQ ID NO: 5 - protein, 31 aa, Homo sapiens
  MISC_FEATURE (8)..(8): Xaa is any amino acid
GPYHPSEXXFTYTTYKIPRQRIMDYYETNSQ

SEQ ID NO: 6 - protein, 28 aa, Homo sapiens
  MISC_FEATURE (5)..(5): Xaa is any amino acid
HPSEXXFTYTTYKIPRQRIMDYYETNSQ

SEQ ID NO: 7 - protein, 11 aa, Homo sapiens
GPYHPSECCFT

SEQ ID NO: 8 - protein, 19 aa, Homo sapiens
TKTESSSRGPYHPSECCFT

SEQ ID NO: 9 - protein, 35 aa, Homo sapiens
TKTESSSQTGGKPKVVKIQLKLVGGPYHPSECCFT

SEQ ID NO: 10 - protein, 14 aa, Homo sapiens
ASLAADTPTACCFS

SEQ ID NO: 11 - protein, 14 aa, Homo sapiens
APMGSDPPTACCFS

SEQ ID NO: 12 - protein, 13 aa, Homo sapiens
SPYSSDTTPCCFA

SEQ ID NO: 13 - protein, 14 aa, Homo sapiens
QPDSVSIPITCCFN

SEQ ID NO: 14 - protein, 12 aa, Homo sapiens
GPASVPTTCCFN

SEQ ID NO: 15 - protein, 68 aa, Homo sapiens
SRGPYHPSECCFTYTTYKIPRQRIMDYYETNSQCSKPGIVFITKRGHSVCTNPSDKWVQDYIKDMKEN

SEQ ID NO: 16 - protein, 67 aa, Homo sapiens
RGPYHPSECCFTYTTYKIPRQRIMDYYETNSQCSKPGIVFITKRGHSVCTNPSDKWVQDYIKDMKEN

SEQ ID NO: 17 - protein, 65 aa, Homo sapiens
PYHPSECCFTYTTYKIPRQRIMDYYETNSQCSKPGIVFITKRGHSVCTNPSDKWVQDYIKDMKEN

SEQ ID NO: 18 - protein, 64 aa, Homo sapiens
YHPSECCFTYTTYKIPRQRIMDYYETNSQCSKPGIVFITKRGHSVCTNPSDKWVQDYIKDMKEN

SEQ ID NO: 19 - protein, 65 aa, Homo sapiens; identical to SEQ ID NO: 17
  MISC_FEATURE (1)..(1): N-terminal proline residue is coupled to nonanoyl

SEQ ID NO: 20 - protein, 7 aa, Unknown
  Substrate peptide for PKC assay; derived from myristoylated alanine-rich protein kinase C substrate protien
FKKSFKL

(19) United States
(12) Patent Application Publication
(10) Pub. No.: US 2009/0082262 A1
(43) Pub. Date: Mar. 26, 2009

(54) Processed human chemokines PHC-1 and PHC-2

(75) Inventors: Wolf-Georg Forssman, Hannover (DE); Michel Detheux, Mons (BE); Marc Parmentier, Beersel (BE); Ludger Standker, Hannover (DE); Frank Kirchhoff, Uln (DE)

Correspondence Address: DARDI & ASSOCIATES, PLLC, 220 S. 6TH ST., SUITE 2000, U.S. BANK PLAZA, MINNEAPOLIS, MN, 55402, US

(73) Assignee: PHARIS BIOTEC GMBH

(21) Appl. No.: 11/580,969

(22) Filed: Oct. 13, 2006

Related U.S. Application Data
(63) Continuation of application No. 10/202,986, filed on Jul. 24, 2002, now abandoned.
Application No. 10/202,986 is a continuation-in-part of application No. 09/891,871, filed on Jun. 22, 2001, now abandoned.
Application No. 09/891,871 is a continuation of application No. PCT/BE00/00128, filed on Oct. 25, 2000.

(30) Foreign Application Priority Data
Oct. 25, 1999 (DE) DE/19951336.8
Jun. 22, 2000 (EP) EP/00870140.1

(51) Int. Cl. A61K 38/16; C07K 14/00

(52) U.S. Cl. 514/12; 530/324

(57) ABSTRACT

The present invention is related to newly identified compounds, polynucleotide sequences encoding the amino acid sequences of the compounds, as well as agonists, antagonists or inhibitors of the compounds for chemokine receptors, especially the CCR-5 receptor and their use in the field of diagnostics and therapeutics involving the chemokine receptors.

### PRIORITY

This application is a continuation of U.S. patent application Ser. No. 10/202,986, filed Jul. 24, 2002, which is a continuation in part of U.S. application Ser. No. 09/891,871, filed Jun. 22, 2001, which is a continuation of PCT/BE00/00128, filed Oct. 25, 2000, which claims priority to EP00870140.1, filed Jun. 22, 2000, and DE 19951336.8, filed Oct. 25, 1999. The contents of each of these documents is incorporated by reference herein in their entirety.

### FIELD OF THE INVENTION

The present invention relates to newly identified compounds, polynucleotide sequences encoding the amino acid sequences of the compounds, agonists as well as antagonists or inhibitors derived from compounds that bind to chemokine receptors and their use in the field of diagnostic and therapeutics involving the chemokine receptors.

### BACKGROUND OF THE INVENTION

Chemokines are small sequences which are known to play a fundamental role in the physiology of acute and chronic inflammatory processes, as well as in the pathological deregulation of these processes.

Furthermore, several chemokine receptors, especially the chemokine receptors identified as CCR5, CXCR4, and CCR3 are known to be involved in HIV viral infection of a patient.

The known chemokines MIP-1α, MIP-1β, RANTES MCP-2 and synthetic compounds derived from these chemokines (AOP-RANTES) have been described as major HIV inhibitory factors.

Furthermore, intensive studies have been performed to screen new compounds (generally synthetic derivatives of natural chemokines and synthetic chemical compounds obtained from library screening) to identify novel compounds exhibiting improved characteristics as viruses antagonists or agonists to chemokine receptors.

### SUMMARY OF THE INVENTION

The present invention aims to, provide new natural compounds, as well as synthetic or natural derivatives thereof, which are antagonists or inhibitors of various chemokine receptors and HIV-1 and HIV-2 co-receptors, especially to the CCR-1 and CCR-5 receptor, and which find application in the treatment and/or the prevention of various diseases.

The present invention is related to a new compound, preferably a chemokine compound which comprises more than 60%, preferably more than 70%, more preferably more than 75%, more than 80%, more than 85%, more than 90% or more than 95% homology (or sequence identity) with SEQ ID NO: 1 (GPYHPSECCFTTYTYKIPRQRIMDYYETNSQCSKPGIVFITKRGHSVCTNPSDKWVQDYI KDMKEN) or SEQ ID NO:2 (HPSECCFTYTFYKIPRQRIMDYYETNSQCSKPGIVFITKRGHSVCTNPSDKWVQDYIKD MKEN), but wherein the compound does not comprise the amino acid sequences presented in SEQ ID NO: 3 (MKISVAAIPFFLLITIALGTKTESSSRGPYHPSECCFTYTTYKIPRQRIMDYYETNSQCSKP G1VFITKRGHSVCTNPSDKWVQDYIKDMKEN) or SEQ ID NO: 4 (TKTESSSRGPYHPSECCFTYTTYKIPRQRIMDYYETNSQCSKPGIVFITKRGHSVCTNPSD KWVQDYIKDMKEN) (HCC-1 peptide), which is an untruncated PHC-1 polypeptide already described in the documents P43443974 and P4427395.9 and by P. Schulz-Knappe et al. (J. Exp. Med., Vol. 183, pp. 295-299 (1999)).

The present invention relates further to a new compound, preferably a synthetic chemokine compound having the sequence of SEQ ID NO: 4, wherein at least six amino acid residues, but not more than 15 amino acid residues are deleted from the N-terminal end of the sequence.

In one embodiment, the compounds having a sequence of SEQ ID NO: 4 with at least six amino acids but not more than 15 amino acids deleted from the N-terminus comprise the sequence of one or more of SEQ ID Nos. 15, 16, 17, 18, or 19.

As used herein a “compound” refers to a molecule, preferably a protein or a nucleic acid encoding a protein. A compound of the invention can be naturally occurring derived by recombinant technology or by other synthetic means known to one skilled in the art. Preferably, a “compound of the invention” is an agonist, antagonist or inhibitor of a chemokine receptor. As used herein a “chemokine compound” refers to a molecule having the amino acid sequence of SEQ ID NO: 4 (HCC-1) wherein at least six, but not more than 15 amino acid residues are deleted from the N-terminus. “Chemokine compounds” useful in the present invention therefore include, but are not limited to PHC-1 (HCC-1[9-74]; SEQ ID NO: 1), PHC-2 (HCC-1[12-74]; SEQ ID NO: 2), HCC-1[7-74] (SEQ ID NO: 15), HCC-1[8-74] (SEQ ID NO: 16), HCC-1[10-74] (SEQ ID NO: 17), and HCC-1[11-74] (SEQ ID NP: 18). “Chemokine compounds” of the present invention include naturally processed human chemokines (PHCs) such as PHC-1 and PHC-2, as well as non-naturally processed, or synthetic human chemokine molecules such as SEQ ID Nos. 15, 16, 17 and 18. As used herein a “chemokine compound” can encompass further, derivatives or analogues of the compound according to the invention are amino acid sequences presented in SEQ ID NO: 1, 2, 15, 16, 17, or 18 coupled with a chemical group at the N-terminal end, such as the nonanoyl-HCC-1[10-74] compound of SEQ ID NO: 19.

As used herein, “homology” refers to the relatedness of two or more separate strands of DNA, based on a comparison of their nucleotide sequences. In general, two polynucleotide sequences that are identical or can each hybridize to the same polynucleotide sequence are homologous. The two sequences are homologous or substantially identical if the sequences each have at least 70%, preferably 80%, more preferably 90% and most preferably 100%, of the same or analogous base sequenced where thymine (T) and uracil (U) are considered the same. Thus, the ribonucleotides A, U, C and G are taken as analogous to the deoxynucleotides dA, dT, dC, and dG, respectively. Homologous sequences can both be DNA or one can be DNA and the other RNA.

Sequence homology (or identity) may be determined using any suitable homology algorithm, using for example default parameters. Advantageously, the BLAST algorithm is employed, with parameters set to default values (Altschul et al., 1997, Nucl. Acids Res. 25: 3389). The BLAST 2.0 program may be set in the following manner to identify database sequences with 100% identity to a test sequence: 1) select the program “blastn” and database “nr”; 2) select the “perform ungapped alignment” check box; 3) enter the sequence in FASTA format; 4) set parameters as follows: Filter, none (this forces the program to use the full length of the test sequence in its alignment; Descriptions, 50; Alignments, 500; Matrix, BLOSUM 62 (default settings); and “Other advanced options,”-e-161; and 5) submit the query. Sequences will be reported in the order of their rank by similarity, and the number of identities at particular nucleotide sites will be indicated for each sequence reported, along with the percent identity. 100% identity over the full length of the sequence indicates that the test sequence is identical to the indicated sequence from the database. The failure of the program to identify sequences with 100% identity indicates the sequence is novel in the GenBank database.

The new compound is preferably an antagonist of the binding of HIV-viruses to chemokine receptors, especially to the CCR-1, CCR-3 and CCR-5 chemokine receptors. Preferably, the new compound is an antagonist that binds to a chemokine receptor and, in particular, binds to the CCR-1, CCR-3 and/or CCR-5 chemokine receptors.

Preferably, the polypeptide according to the invention, is the chemokine PHC-1 or PHC-2 having the amino acid sequence presented in SEQ ID NO: 1 and SEQ ID NO: 2, respectively or biologically active fragments or portions thereof.

As used herein, “biologically active” refers to a polypeptide, a derivative, a fragment or a portion thereof, that has normal or near normal pharmacology (e.g. receptor binding activity, the response to an activator or an inhibitor, or binding to a nucleic acid or binding protein are at least 90% of the level of activity, response or binding exhibited by the corresponding wild-type or complete polypeptide).

The active fragments or portions thereof are advantageously NH₂-terminal amino acid sequences of at least 10, 15, 20, 25, or 30 amino acids, preferably at least 40 amino acids, more preferably at least 50 or 55 amino acids, of the original above-described complete sequences presented in SEQ ID NO: 1 or SEQ ID NO: 2, which may include the deletion of one or more amino acids; as well as their derivatives, or in particular compounds having at least 10, 15, 20, 25, 30, 40, 50 or 55 amino acids of the complete amino acid sequences presented in SEQ ID NO: 1 or SEQ ID NO: 2 with one or more additional amino acid residue(s) in the sequence, possibly linked to (for example by covalent or hydrogen bonding or electrostatic attraction or modified by (substitution of one or more carbon atoms or one or more alkyl) amide, acetyl, phosphoryl and/or glycosyl or other substitution groups.

The modification of the original amino acid sequences presented in SEQ ID NO: 1 or SEQ ID NO: 2 preferably occurs at the C-terminal end by fusion with other amino acid sequences, tags, or the incorporation of the above-identified groups including fluorescent groups upon one or both extremities of the original sequence presented in SEQ ID NO: 1 in order to provide a substrate for proteolytic activity screening. As used herein, “tagged” refers to a polypeptide that is covalently or non-covalently attached to a tag or detectable moiety including but not limited to his, hemagglutinin, myc, FLAG, GST or calmodulin binding peptides. “Tagging”, as used herein, refers to a fusion of short peptides or protein domains. Among the polypeptide and their derivatives (analogues) are excluded the compounds having the amino acid sequences presented in SEQ ID NO: 3 or SEQ ID NO: 4 and their active amidated, acetylated, phosphorylated and/or glycosylated derivatives, which is the compound which does not present the activity of the compound according to the invention, and which is not able to bind to the following chemokine receptors CCR-1, CCR-3, CXR-4 and/or CCR-5.

Preferably, additional amino acid residues added to the N-terminal or C-terminal portion of the sequence presented in SEQ ID NO: 1, SEQ ID NO: 2, or a chemokine compound as described herein, are chains of 2 to 10 amino acids.

According to a preferred embodiment of the present invention, the derivatives or analogues of the compound according to the invention are amino acid sequences presented in SEQ ID NO: 1, 2, 15, 16, 17, or 18 coupled with a chemical group at the N-terminal end. A non-limiting example of such a derivative is the Nonanoyl-HCC-1 [10-74] compound of SEQ ID NO: 19.

In the following description, the peptides according to the invention are identified as PHC chemokines or PHC derivatives or analogues, or chemokine compound derivatives or analogs, which correspond to chemokine compounds wherein a portion, preferably the N-terminus extremity, is modified by its coupling to or its substitution with a chemical group, preferably according to the method described by H. Gaertner et al., (J. Biol. Chem., Vol. 271, pp. 2591-2603 (1996) and G. Simmons et al. (Science, Vol. 276, pp. 276-279 (1997)).

Preferably, said PHC derivatives or analogues have one of the following structures:

[Glyoxyloyl¹]PHC 1-Pentane oxime,
[Glyoxyloyl¹) PHC Caproyl oxime,

### D-Met-[Gly¹]PHC,

### L-Pyrollidone Carboxoyl-[Gly¹] PHC,

### [Glyoxyloyl¹]PHC,

[Glyoxyloyl¹] PHC 1-acetyl-ethylamine-2oxime,
[Glyoxyloyl¹] PHC S-Methyl 1-Thiopropane-3-oxime,
[Glyoxyloyl¹]PHC 2-Pentene oxime,
[Glyoxyloyl¹]PHC Methane oxime,

### Hexanoyl-[Gly¹]PHC,

[Glyoxyloyl¹] PHC Phenylmethane oxime,
[Glyoxyloyl¹]PHC 1-Propane oxime,
[Glyoxyloyl¹]PHC 1-Butane oxime,
[Glyoxyloyl¹]PHC 2-Butane oxime,

### Hexanyl-[Gly¹]PHC,

[Glyoxyloyl¹]PHC 2-Propane oxime,
[Glyoxyloyl¹]PHC Pentane oxime,

### Nonanyl-PHC,

[Glyoxyloyl¹] PHCPHC 2-Heptane oxime,
[Glyoxyloyl¹]PHC Ethane oxime,
[Glyoxyloyl¹]PHC 1-Heptane oxime,
[Glyoxyloyl¹]PHC 1-hexane oxime,
[Glyoxyloyl¹]PHC 1-Pentene oxime,

### Nonanoyl-PHC,

or are a compound having one of the following formulas:

CH₃—(CH₂)₄—CO—NH—CH₂—CO—PHC,

CH₃—(CH₂)₅—H—CH₂—CO—PHC,

CH₃—(CH₂)₇)—CO—PHC,

CH₃—(CH₂)₈)—PHC,

HOOC—(CH₂)₅—O—N═CH—CO—PHC.

Analogues of the compounds of the invention are also molecules such as antibodies or other products obtained by recombinant chemistry or library screening which may mimic and preferably increase the interactions of said compounds to their receptors.

According to another preferred embodiment of the present invention, one or more amino acids, preferably a lysine, histidine, glutamate, aspartate, or cysteine residue of the PHC peptides are modified by a coupling with a chemical group having the structure of a polyethyleneglycol. Such modification allows an increasing of the plasma half-life time of the original PHC molecule.

Furthermore, the compound according to the invention, for diagnostic purposes, comprises a detectable label upon one or more of its extremities. Detectable labels include but are not limited to fluorescent compounds, isotopic compounds, chemiluminescent compounds, quantum dot labels, biotin, enzymes, electron-dense reagents, and haptens or proteins for which antisera or monoclonal antibodies are available. The various means of detection include but are not limited to spectroscopic, photochemical, radiochemical, biochemical, immunochemical, or chemical means.

The preferred PHC chemokine compounds according to the invention are of human origin and can be obtained by an isolation procedure departing from human blood ultrafiltrate (hemofiltrate) and by using biological assay systems to determine the biological activity of the compound.

As shown in FIG. 1 and in order to achieve the purification of the compound according to the invention, peptides are prepared from human hemofiltrate as described by Schulz-Knaap et al, J. Chrom. A., Vol. 776, pp. 125-132 (1997). Thereafter, the obtained hemofiltrate fractions are screened for their chemokine receptor(s) stimulatory activity, and the biologically active fractions are further purified by chromatography procedures using diverse reverse phase column chromatographic steps as described in Example 1.

In one embodiment, the chemokine compounds are cleaved from a molecule having an amino acid sequence comprising SEQ ID NO: 4 by an enzyme. Preferably the enzyme is streptokinase or urokinase.

The biologically active peptides obtained by the chromatographic purification are subjected to a structure determination, including mass spectrometry, and peptide sequence analysis.

However, such compounds can also be obtained by recombinant genetic technologies or by synthesis, said methods possibly comprising also purification steps that can be carried out by one skilled in the art. As used herein, the term “synthetic” when used in reference to a peptide, polypeptide or polynucleotide means that the amino acid or nucleotide subunits were chemically joined in vitro without the use of cells or polymerizing enzymes. The chemistry of polynucleotide and peptide synthesis is well known in the art.

The invention also relates to a chemokine compound comprising the amino acid sequence presented in SEQ ID Nos. 1, 2, 15, 16, 17, or 18, or a biologically active fragment or portion thereof.

As used herein, a “chemokine” refers to a type of cytokine (a soluble molecule that a cell produces to control reactions between other cells). In certain embodiments, a chemokine specifically alters the behavior of leukocytes (white blood cells). Examples of chemokines include MIP-1α, MIP-β, RANTES, MCP-2, interleukin 8, platelet factor 4, melanoma growth stimulatory protein and the like.

In another preferred embodiment, the chemokine compound comprises the amino acid sequence presented in SEQ ID Nos. 1, 2, 15, 16, 17, or 18, or a biologically active fragment or portion thereof, (i.e., having the same biological activity as the full length sequence) modified by or linked to at least one of an amide, acetyl, phosphoryl, or glycosyl group.

In another preferred embodiment, the compound binds to at least one of the chemokine receptors selected from the group consisting of CCR-1, CCR-3 and CCR-5.

As used herein, “binding” or “association” refers to a polypeptide (for example a receptor) and a ligand having a binding constant sufficiently strong to allow detection or binding by a detection means that is appropriate for a detectable ligand that is labeled (for example FRET, autoradiography, western blot analysis, FACS, gel shift analysis etc. . . . ) or by a biological assay, or a messenger assay as described herein, wherein the polypeptide and ligand are in physical contact with each other and have a dissociation constant (Kd) of about 10 μM or lower.

A second aspect of the present invention is related to a polynucleotide comprising at least a sequence encoding the compound according to the invention, preferably a polynucleotide sequence encoding the compound or its active portions having the amino acid sequence presented in SEQ ID NO: 1 or SEQ ID NO: 2 or a biologically active fragment or portion thereof presenting the activity of the full sequence.

As used herein, “presenting the activity of the full sequence” refers to having normal or near normal pharmacology (e.g. receptor binding activity, the response to an activator or an inhibitor, or binding to a nucleic acid or binding protein are at least 90% of the level of activity, response or binding exhibited by the corresponding wild-type or complete polypeptide).

Another aspect of the present invention is related to novel antagonists or inhibitors of the compounds of the invention or the polynucleotide encoding the amino acid sequence of the compounds of the invention, wherein the antagonist or inhibitor binds to a chemokine receptor, preferably a receptor selected from the group consisting of the CCR-1, CCR-3 and CCR-5 receptors, preferably to the CCR-5 receptor. Preferably, the antagonist or inhibitor is not a natural known compound.

As used herein, a “natural known compound” refers to a naturally occurring compound that is known in the art. An antagonist or inhibitor that is “not a natural known compound” includes a naturally occurring compound that is not known in the art or is a compound that is produced by recombinant technology or by synthetic methods.

An antagonist of the compound according to the invention means a molecule or a group of molecules able to bind to the receptor and block the binding of the compound according to the invention to the receptor. The unknown antagonist is also an antagonist to a known “natural” compounds, including micro-organisms such as bacteria, protozea, viruses or portions thereof (that bind to a chemokine receptor), in particular immunodeficiency viruses 1 and/or 2 (HIV-1 and/or HIV-2) or other viruses affecting a patient. Preferably, said viruses are selected from the group consisting of herpes simplex virus, varicella zoster virus, hepatitis-A viruses, hepatitis-B viruses, zytomegalo virus, influenza virus, polio virus, rhino virus, measles virus, German measles virus, rabies virus, Rous sarcoma virus, Pox virus and Epstein-Barr virus.

The known “natural” compound may also be a specific portion of a virus that is able to bind to a chemokine receptor. Examples of such portions are the GP 120/160 glycoprotein of HIV-1 and/or HIV-2 which is known to interact specifically with the CCR-5 receptor.

As used herein, an “antagonist” also encompasses a ligand which competitively binds to the receptor at the same site as an agonist, but does not activate an intracellular response initiated by an active form of a receptor, and thereby inhibits the intracellular response induced by an agonist by at least 10%, preferably 15-25%, more preferably 25-50% and most preferably, 50-100%, as compared to the intracellular response in the presence of an agonist and in the absence of an antagonist. An antagonist does not diminish the base line intracellular response that occurs in the absence of an agonist.

An inhibitor of the compound according to the invention is a molecule that binds to a compound of the invention or a nucleotide sequence encoding the compound, and, preferably, blocks the binding and interaction of the compound to other molecules, including the receptor according to the invention. As used herein, an “inhibitor” refers to a molecule that decreases binding of a compound to a receptor by at least 10-15%, preferably 15-30%, more preferably 30-75% and most preferably 75%-100%, as compared to the amount of binding in the absence of an inhibitor.

Examples of an inhibitor of the invention are (monoclonal or polyclonal) antibodies or hypervariable portions of said antibodies that are unlabeled or labeled. The antibody (monoclonal or polyclonal) or its hypervariable portion (Fab′, Fab2′, etc. . . . ) as well as the hybridoma cell producing the antibody are a further aspect of the present invention, which finds specific industrial application in the field as diagnostic tools, especially for the monitoring of the effect of the compound of the invention upon chemokine receptors, especially CCR-1, CCR-3 and/or CCR-5 receptors.

In one embodiment, the inhibitor of a compound of the invention binds to a receptor selected from the group consisting of CCR-1, CCR-3 and CCR-5, is not a natural known compound and further comprises an antisense oligonucleotide or ribozyme having a sequence of at least 15 nucleotides capable of specifically hybridizing to an mRNA molecule encoding the compound of the invention and preventing the translation of the mRNA molecule. In another embodiment, the inhibitor of a compound of the invention binds to a receptor selected from the group consisting of CCR-1, CCR-3 and CCR-5, is not a natural known compound and further comprises an antisense oligonucleotide capable of specifically hybridizing to a DNA molecule encoding a compound of the invention.

As used herein, a “ribozyme” refers to an RNA molecule that has catalytic activity.

As used herein, “antisense” refers to a complementary RNA sequence that binds to an blocks the transcription of a naturally-occurring (sense) mRNA molecule.

As used herein, “nucleic acid hybridization” refers to hydrogen bonding between two complementary nucleic acids sequences. As used herein, “specifically hybridized” refers to a pair of nucleic acid sequences that associate with each other with a dissociation constant (K_{D}) of at least about 1×10³M⁻¹, usually at least 1×10⁴M⁻¹, typically at least 1×10⁵M⁻¹, and preferably at least 1×10⁶M-Ito 1×10⁷M⁻¹ or more.

Stringency of hybridization refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency refers to conditions that permit hybridization of only those nucleic acid sequences that form stable hybrids in 1 M Na+ at 65-68° C. High stringency conditions can be provided, for example, by hybridization in an aqueous solution containing 6× SSC, 5×Denhardt's, 1% SDS (sodium dodecyl sulphate), 0.1 Na+ pyrophosphate and 0.1 mg/ml denatured salmon sperm DNA as a non specific competitor. Following hybridization, high stringency washing may be done in several steps, with a final wash (about 30 min) at the hybridization temperature in 0.2-0.1×SSC, 0.1% SDS.

Moderate stringency refers to conditions equivalent to hybridization in the above described solution but at about 60-62° C. In that case the final wash is performed at the hybridization temperature in 1×SSC, 0.1% SDS.

Low stringency refers to conditions equivalent to hybridization in the above described solution at about 50-52° C. In that case, the final wash is performed at the hybridization temperature in 2× SSC, 0.1% SDS.

It is understood that these conditions maybe adapted and duplicated using a variety of buffers, e.g. formamide-based buffers, and temperatures. Denhardt's solution and SSC are well known to those of skill in the art as are other suitable hybridization buffers (see e.g. Sambrook et al., eds. (1989) Molecular Cloning: A Laboratory Manual, Cold Spring Harbor Laboratory Press, New York or Ausubel et al., eds. (1990) Current Protocols in Molecular Biology, John Wiley & Sons, Inc.). Optimal hybridization conditions have to be determined empirically, as the length and the GC content of the hybridizing pair also play a role.

Typically, specific hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference.

Numerous factors influence the efficiency and selectivity of hybridization of a first nucleic acid to a second nucleic acid molecule. These factors, which include nucleic acid length, nucleotide sequence and/or composition, hybridization temperature, buffer composition and potential for steric hindrance in the region to which the primer is required to hybridize, will be considered when designing oligonucleotides according to the invention.

A positive correlation exists between nucleic acid length and both the efficiency and accuracy with which a first nucleic acid will anneal to a second nucleic acid. In particular, longer sequences have a higher melting temperature (T_{M}) than do shorter ones, and are less likely to be repeated within a given target sequence, thereby minimizing promiscuous hybridization. Nucleic acid sequences with a high G-C content or that comprise palindromic sequences tend to self-hybridize, as do their intended target sites, since unimolecular, rather than bimolecular, hybridization kinetics are generally favored in solution. However, it is also important to design a nucleic acid that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with nucleic acid annealing efficiency, as does the concentration of organic solvents, e.g. formamide, that might be included in a hybridization mixture, while increases in salt concentration facilitate binding. Under stringent annealing conditions, longer hybridization probes, or synthesis primers, hybridize more efficiently than do shorter ones, which are sufficient under more permissive conditions. Preferably, stringent hybridization is performed in a suitable buffer (for example, 1× Sentinel Molecular Beacon PCR Core buffer, Stratagene Catalog #600500; 1× Pfu buffer, Stratagene Catalog #200536; or 1× Cloned Pfu buffer, Stratagene Catalog #200532) under conditions that allow the first nucleic acid sequence to hybridize to the second nucleic acid sequence (e.g., 95° C.). Stringent hybridization conditions can vary (for example, salt concentrations may range from less than about 1M, more usually less than about 500 mM and preferably less than about 200 mM) and hybridization temperatures can range (for example, from as low as 00 C to greater than 220 C, greater than about 300 C, and (most often) in excess of about 370 C), depending upon the length and/or nucleic acid composition of the nucleic acids. Longer fragments may require higher hybridization temperatures for specific hybridization. As several factors affect the stringency of hybridization, the combination of parameters is more important than the absolute measure of a single factor.

As used herein, “preventing translation of an mRNA” refers to inhibiting the amount of protein produced from an mRNA by at least 10%, preferably 15-25%, more preferably 25-50% and most preferably 50-100%, as compared to the amount of protein produced by an mRNA in the absence of an agent that prevents translation (for example an antisense oligonucleotide or a ribozyme).

In another preferred embodiment, the antisense oligonucleotide comprises at least one chemical analogue of a nucleotide. According to the invention, the nucleic acid may be chemically or biochemically modified or may contain non-natural or derivatized nucleotide bases. Such modifications include, for example, labels, methylation, substitution of one or more of the naturally occurring nucleotides with an analog, internucleotide modifications such as uncharged linkages (e.g. methyl phosphonates, phosphorodithioates, etc.), pendent moieties (e.g., polypeptides), intercalators, (e.g. acridine, psoralen, etc.) chelators, alkylators, and modified linkages (e.g. alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As mentioned above, the present invention is also related to the polynucleotide encoding the amino acid sequence of the compound according to the invention (such as a cDNA molecule, genomic DNA molecule or RNA molecule).

Another aspect of the present invention is related to a vector comprising the polynucleotide, preferably a vector adapted for expression in a cell, and which comprises the regulatory element necessary for expression of the polynucleotide in a cell (preferably a cell selected from the group consisting of a bacterial cell, a yeast cell, an insect cell or a mammalian cell).

The vector could be a plasmid or a virus, preferably a baculovirus, an adenovirus, a retrovirus or a semliki forest virus.

Another aspect of the present invention is related to a cell transformed (according to known techniques by the one skilled in the art) by the vector of the invention, preferably a mammalian cell, (such as a cell selected from the group consisting of COS-7 cell, CHO-K1 cell, LM(tk-) cell, NIH-3T3 cell, HEK-293 cell or K-562 cell).

The inhibitor of the compound of the invention can also be a nucleic acid probe of more than 15, 20, 25 or 30 nucleotides, such as an antisense oligonucleotide having a sequence capable of specifically hybridising to an mRNA molecule encoding the compound of the invention, so as to prevent translation of the mRNA molecule or an antisense oligonucleotide or a ribozyme capable of specifically hybridising to a DNA molecule encoding the compound according to the invention. In certain embodiments, the antisense oligonucleotide further comprises chemical analogues of nucleotides of the oligonucleotide.

The present invention is also related to a transgenic non human mammal comprising a homologous recombination “knock-out” of the polynucleotide according to the invention or a transgenic non human mammal overexpressing (that is, expressing at a level that is greater then the natural level of expression of the polynucleotide) the compound according to the invention. Such transgenic non-human mammals can be obtained by methods well known by those skilled in the art, for instance as described in the document W098/20112 using the classical techniques based upon the transfection of embryonic stem cells preferably according to the method described by Carmeliet et al. (Nature, Vol. 380, pp. 435-439 (1996)).

Preferably, said transgenic non human mammal overexpressing the polynucleotide according to the invention comprises a polynucleotide incorporated into a DNA construct with an inducible promoter allowing the overexpression of the compound according to the invention.

As used herein, an “inducible promoter” refers to a promoter that is only expressed in the presence of an exogenous or endogenous chemical (for example an alcohol, a hormone, or a growth factor), or in response to developmental changes or at particular stages of differentiation.

Possibly, said nucleic acid construct also comprises tissue and specific regulatory elements.

As used herein, “tissue regulatory elements” refers to nucleic acid sequences that regulate the expression of a gene such that it is only expressed in a particular tissue.

As used herein, “regulatory elements” refers to refers to nucleic acid sequences that regulate the expression of a gene such that it is only expressed in a particular tissue, at a particular stage of development or differentiation, in the presence of an exogenous or endogenous chemical or factor (i.e., DNA binding protein) or at a particular temperature.

The invention encompasses a method of identifying an agent that modulates the function of a chemokine receptor, the method comprising: a) contacting a chemokine receptor polypeptide with a chemokine compound of the invention in the presence and absence of a candidate modulator under conditions permitting the binding of the chemokine compound to the chemokine receptor polypeptide; and b) measuring the binding of the chemokine receptor to the chemokine compound, wherein a decrease in binding in the presence of the candidate modulator, relative to the binding in the absence of the candidate modulator, identifies the candidate modulator as an agent that modulates the function of the chemokine receptor.

In a preferred embodiment, the chemokine receptor is selected from the group consisting of CCR-1, CCR-3, and CCR-5.

In a preferred embodiment of either of the preceding methods, the measuring is performed using a method selected from label displacement, surface plasmon resonance, fluorescence resonance energy transfer, fluorescence quenching, and fluorescence polarization.

The invention further encompasses a method of identifying an agent that modulates the function of chemokine receptor, the method comprising: a) contacting a chemokine receptor with a chemokine compound in the presence and absence of a candidate modulator; and b) measuring a signaling activity of the chemokine receptor, wherein a change in the activity in the presence of the candidate modulator relative to the activity in the absence of the candidate modulator identifies the candidate modulator as an agent that modulates the function of the chemokine receptor.

In a preferred embodiment of each of the preceding methods, the chemokine compound is detectably labeled. It is preferred that the chemokine compound is detectably labeled with a moiety selected from the group consisting of a radioisotope, a fluorophore, a quencher of fluorescence, an enzyme, an affinity tag, and an epitope tag.

In one embodiment of any of the preceding methods, the contacting is performed in or on a cell expressing the chemokine receptor.

In another embodiment of any of the preceding methods, the method is performed using a membrane fraction from cells expressing the chemokine receptor.

In another embodiment, the candidate modulator is selected from the group consisting of a peptide, a polypeptide, an antibody or antigen-binding fragment thereof, a lipid, a carbohydrate, a nucleic acid, and a small organic molecule.

In another embodiment, the step of measuring a signaling activity of the chemokine receptor comprises detecting a change in the level of a second messenger.

In another embodiment, the step of measuring a signaling activity comprises measurement of guanine nucleotide binding or exchange, adenylate cyclase activity, cAMP, Protein Kinase C activity, phosphatidylinosotol breakdown, diacylglycerol, inositol triphosphate, intracellular calcium, arachadonic acid, MAP kinase activity, tyrosine kinase activity, or reporter gene expression.

As used herein, the term “chemokine receptor activity” refers to specific binding of a chemokine compound or a functional fragment thereof by a chemokine receptor polypeptide.

As used herein, the term “chemokine receptor signaling activity” refers to the initiation or propagation of signaling by a chemokine receptor. Chemokine receptor signaling activity is monitored by measuring a detectable step in a signaling cascade by assaying one or more of the following: stimulation of GDP for GTP exchange on a G protein; alteration of adenylate cyclase activity; protein kinase C modulation; phosphatidylinositol breakdown (generating second messengers diacylglycerol, and inositol triphosphate); intracellular calcium flux; activation of MAP kinases; modulation of tyrosine kinases; or modulation of gene or reporter gene activity. A detectable step in a signaling cascade is considered initiated or mediated if the measurable activity is altered by 10% or more above or below a baseline established in the substantial absence of a chemokine compound relative to any of the chemokine receptor activity assays described herein below. The measurable activity can be measured directly, as in, for example, measurement of cAMP or diacylglycerol levels. Alternatively, the measurable activity can be measured indirectly, as in, for example, a reporter gene assay.

As used herein, the term “detectable step” refers to a step that can be measured, either directly, e.g., by measurement of a second messenger or detection of a modified (e.g., phosphorylated) protein, or indirectly, e.g., by monitoring a downstream effect of that step. For example, adenylate-cyclase-activation results in the generation of cAMP. The activity of adenylate cyclase can be measured directly, e.g., by an assay that monitors the production of cAMP in the assay, or indirectly, by measurement of actual levels of cAMP.

As used herein, the term “isolated” refers to a population of molecules, e.g., polypeptides or polynucleotides, the composition of which is less than 50% (by weight), preferably less than 40% and most preferably 2% or less, contaminating molecules of an unlike nature.

As used herein, the terms “candidate compound” and “candidate modulator” refer to a composition being evaluated for the ability to modulate ligand binding to a chemokine receptor polypeptide or the ability to modulate an activity of a chemokine receptor polypeptide. Candidate modulators can be natural or synthetic compounds, including, for example, small molecules, compounds contained in extracts of animal, plant, bacterial or fungal cells, as well as conditioned medium from such cells.

As used herein, the term “small molecule” refers to a compound having molecular mass of less than 3000 Daltons, preferably less than 2000 or 1500, still more preferably less than 1000, and most preferably less than 600 Daltons. A “small organic molecule” is a small molecule that comprises carbon.

As used herein, the term “conditions permitting the binding of a chemokine compound to a chemokine receptor” refers to conditions of, for example, temperature, salt concentration, pH and protein concentration under which a chemokine compound binds a chemokine receptor. Exact binding conditions will vary depending upon the nature of the assay, for example, whether the assay uses viable cells or only membrane fraction of cells. However, because chemokine receptors useful in the present invention are cell surface proteins, and because chemokine compounds are secreted polypeptides that interacts with a chemokine receptor on the cell surface, favored conditions will generally include physiological salt (90 mM) and pH (about 7.0 to 8.0). Temperatures for binding can vary from 15° C. to 37° C., but will preferably be between room temperature and about 30° C. The concentration of a chemokine compound and chemokine receptor polypeptide in a binding reaction will also vary, but will preferably be about 0.1 pM (e.g., in a reaction with radiolabeled tracer chemokine compound, where the concentration is generally below the K_{d}) to 1 μM (e.g., chemokine compound as competitor). As an example, for a binding assay using chemokine receptor-expressing cells and purified, recombinant, labeled chemokine compound polypeptide, binding is performed using 0.1 mM labeled chemokine compound, 100 nM cold chemokine compound, and 25,000 cells at 27° C. in 250 μl of a binding buffer consisting of 50 mM HEPES (pH 7.4), 1 mM CaCl₂, and 0.5% Fatty acid free BSA.

As used herein, the term “membrane fraction” refers to a preparation of cellular lipid membranes comprising a chemokine receptor polypeptide. As the term is used herein, a “membrane fraction” is distinct from a cellular homogenate, in that at least a portion (i.e., at least 10%, and preferably more) of non-membrane-associated cellular constituents has been removed. The term “membrane associated” refers to those cellular constituents that are either integrated into a lipid membrane or are physically associated with a component that is integrated into a lipid membrane.

As used herein, the term “decrease in binding” refers to a decrease of at least 10% in the binding of a chemokine compound polypeptide or other agonist to a chemokine receptor polypeptide as measured in a binding assay as described herein.

As used herein, the term “increase in binding” refers to an increase of at least 10% in the binding of a chemokine compound polypeptide or other agonist to a chemokine receptor polypeptide as measured in a binding assay as described herein.

As used herein, the term “second messenger” refers to a molecule, generated or caused to vary in concentration by the activation of a cell surface receptor, that participates in the transduction of a signal from that receptor. Non-limiting examples of second messengers include cAMP, diacyiglycerol, inositol triphosphates and intracellular calcium. The term “change in the level of a second messenger” refers to an increase or decrease of at least 10% in the detected level of a given second messenger relative to the amount detected in an assay performed in the absence of a candidate modulator.

As used herein, the term “binding” refers to the physical association of a ligand (e.g., a chemokine compound polypeptide) with a receptor (e.g., a chemokine receptor). As the term is used herein, binding is “specific” if it occurs with an EC₅₀ or a K_{d} of 100 nM or less, generally in the range of 100 nM to 10 pM. For example, binding is specific if the EC₅₀ or K_{d} is 100 nM, 50 nM, 10 nM, 1 nM, 950 pM, 900 pM, 850 pM, 800 pM, 750 pM, 700 pM, 650 pM, 600 pM, 550 pM, 500 pM, 450 pM, 400 pM, 350 pM, 300 pM, 250 pM, 200 pM, 150 pM, 100 pM, 75 pM, 50 pM, 25 pM or 10 pM or less.

As used herein, an “agonist” refers to a molecule which activates an intracellular response when it binds to a receptor. An agonist may alternatively enhance GTP binding to membranes when it binds to a receptor. An agonist, according to the invention may increase internalization of a cell surface receptor such that the cell surface expression of a receptor is decreased by at least 2-fold, preferably 5-fold, more preferably 10-fold and most preferably, 100-fold or more (i.e., 150-fold, 200-fold, 250-fold, 500-fold, 1000-fold, 10,000-fold etc. . . . ), as compared to the number of cell surface receptors present on the surface of a cell in the absence of an agonist. In another embodiment of the invention, an agonist stabilizes a cell surface receptor and increases the cell surface expression of a receptor by at least 2-fold, preferably 5-fold, more preferably 10-fold and most preferably, 100-fold or more (i.e., 150-fold, 200-fold, 250-fold, 500-fold, 1000-fold, 10,000-fold etc. . . . ), as compared to the number of cell surface receptors present on the surface of a cell in the absence of agonist.

The invention also provides for an antagonist or inhibitor identified by the methods of the invention.

The invention also provides for an agent that regenerates a compound of the invention.

As used herein, “agent” refers to a molecule, preferably a protein or nucleic acid molecule that is naturally occurring or is produced by recombinant methods or by synthetic methods known in the art. Preferably, an “agent” can increase the amount of a compound, either in vitro or in vivo, by at least 2-fold, preferably 2-10-fold, more preferably 10-50-fold and most preferably, 50-fold or more, as compared to the amount of compound that is present in the absence of the agent. An “agent” according to the invention can be a protease that cleaves a larger precursor polypeptide to produce a compound of the invention.

As used herein, “regenerate” refers to produce or synthesize. In certain embodiments, a compound is regenerated by cleaving a larger precursor polypeptide to produce the compound.

In a preferred embodiment, the agent is a protease that cleaves a polypeptide comprising the amino acid sequence presented in SEQ ID NO:3 or SEQ ID NO:4 to a compound comprising the amino acid sequence presented in SEQ ID NO: 1 or SEQ ID NO:2. In one embodiment, the agent is capable of cleaving the sequence of SEQ ID NO: 3 or 4 to produce the sequence of SEQ ID Nos 15, 16, 17, or 18.

In another preferred embodiment, the agent further comprises recombinant urokinase or streptokinase.

The invention also provides for an inhibitor or activator of an agent that increases or decreases the amount of regeneration of a compound of the invention by the agent.

An “inhibitor of an agent” refers to a molecule, for example a protein or nucleic acid, that decreases the amount of a compound (by at least 5%, preferably 5-25%, more preferably 25-50% and most preferably 50-100%) that is regenerated by an agent in the absence of the inhibitor of the agent. An “inhibitor of an agent” can also decrease the rate at which an agent regenerates a compound of the invention (by at least 5%, preferably 5-25%, more preferably 25-50% and most preferably 50-100%) as compared to the rate of regeneration of a compound by the agent in the absence of the inhibitor of the agent.

An “activator of an agent” refers to a molecule, for example a protein or nucleic acid, that increases the amount of a compound (by at least 2-fold, preferably 2-10-fold, more preferably 10-50-fold and most preferably, 50-fold or more, for example 100, 1000, 10,000-fold) that is regenerated by an agent as compared to the amount of compound that is regenerated by an agent in the absence of an activator of an agent. An “activator of an agent” also refers to a molecule, for example a protein or nucleic acid, that increases the rate of regeneration of a compound (by at least 2-fold, preferably 2-10-fold, more preferably 10-50-fold and most preferably, 50-fold or more, for example 100, 1000, 10,000-fold) that is regenerated by an agent as compared to the rate of regeneration of a compound by an agent in the absence of an activator of an agent.

Another aspect of the present invention is related to a pharmaceutical composition (vaccine), comprising an effective amount of one or more chemokine compounds according to the invention, their analogues, antagonists or inhibitors (directed against said compounds), as well as the polynucleotides encoding the compounds, agents of the invention, and inhibitors or activators of an agent of the invention, and a pharmaceutically adequate carrier or diluent for use as a medicament.

Advantageously, another aspect is related to the use of one or more of the chemokine compounds, analogues, antagonists or inhibitors and polynucleotides for the manufacture of a medicament in the treatment and/or the prevention of various diseases (wherein CCR-1, CCR-3 and CCR-5 are involved), especially viral infections, in particular diseases (AIDS) induced by a human immunodeficiency virus 1 and/or 2 (HIV-1 and/or HIV-2) or the other viruses described above; as well as for the preparation of a medicament for the treatment and/or the prevention of disturbances of cell migration, diseases or perturbations of the immune system, including cancers, development of tumours and tumour metastasis and Hodgkins lymphoma, rheumatoid arthritis, psoriasis, chronic contact dermatitis, inflammatory bowel disease, multiple sclerosis (MS), stroke, sarcoidosis, organ transplant rejection, inflammatory and neoplastic processes, viral, bacterial and fungal infections, for wound and bone healing and dysfunctions of regulatory growth functions, pain, diabetes, obesity, anorexia, bulimia, Parkinson's disease, acute heart failure, hypotension, hypertension, urinary retention, osteoporosis, angina pectoris, myocardial infarction, restenosis, atherosclerosis, diseases characterised by excessive smooth muscle cell proliferation, aneurysms, wound healing, diseases characterised by loss of smooth muscle cells or reduced smooth muscle cell proliferation, stroke, ischemia, ulcers, allergies (including asthma), benign prostatic hypertrophy, migraine, vomiting, psychotic and neurological disorders, including anxiety, schizophrenia, manic depression, depression, delirium, dementia and severe mental retardation, degenerative diseases, neurodegenerative diseases such as Alzheimer's disease, and dyskinasias such as Huntington's disease or Gilles de la Tourett's syndrome among others.

Another aspect of the present invention is related to a diagnostic kit or device comprising the (possibly labelled) chemokine compounds, analogues, antagonists and/or inhibitors, agents and polynucleotides according to the invention, and packaging means.

A preferred embodiment of the diagnostic kit or device is a kit or device, for the monitoring of the activity of the chemokine compounds upon various chemokine receptors, especially the monitoring of their activity with a correlation to their therapeutic or prophylactic action upon one or more of the above mentioned diseases or symptoms of the diseases, and packaging means.

According to another preferred embodiment of the present invention, the diagnostic kit or device is a high throughput screening diagnostic and/or dosage device, intended for high throughput identification, recovering and/or dosage of such chemokine compounds, analogues, antagonists or inhibitors which allow the identification of high amounts of compounds (known or unknown), acting as antagonists or inhibitors of the compounds according to the invention.

Preferably, the high throughput screening diagnostic and/or dosage device is based upon the method described in the international patent application WO 00/02045.

The high throughput screening technology can be performed on various solid supports, such as microtiter plates or biochips according to techniques known to the one skilled in the art.

The present invention is also related to the molecule characterised and possibly recovered by the methods of the invention, to the molecule identified by the methods of the invention and to a pharmaceutical composition (vaccine) comprising the molecule of the invention and a pharmaceutically acceptable carrier or diluent.

Another aspect of, the present invention is related to a molecule, possibly a recombinant molecule, preferably a protease including a recombinant protease, such as an urokinase or staphylokinase, responsible for the generation of the chemokine compounds (preferably, the molecule is responsible for the generation of PHC-1 and PHC-2, identified as SEQ ID NO: 1 and SEQ ID NO: 2 generated naturally or artificially from SEQ ID NO: 3 or SEQ ID NO: 4), as well as inhibitors or activators of the molecule for providing advantageously a treatment or a prevention of the associated diseases, especially the diseases described above. Such molecules, including recombinant molecules having possibly increased enzymatic activity (obtained and defined by known screening techniques), are advantageously administered according to known techniques by the person skilled in the art to a patient in order to obtain the cleavage of the molecule presented in SEQ ID NO: 3 or SEQ ID NO: 4 into the specific PHC-1 and PHC-2 compounds according to the invention, and possibly their modification into improved derivatives. The method of treatment of the above-identified diseases or the symptoms associated with the above-identified diseases is obtained by using a sufficient amount of the molecules, preferably the proteases, which will be administered to a patient who may suffer from the above-identified diseases or symptoms, in order to allow or increase (facilitate) the cleavage of the compound corresponding to SEQ ID NO: 3 or SEQ ID NO: 4 into the compounds according to the invention.

Preferably, the pharmaceutical composition according to the invention is also a nucleotide sequence encoding the above-identified recombinant proteases for an in vivo treatment or for a-treatment by a cell for an ex vivo treatment. The cell will be readministered to a patient to obtain a genetic therapy or prophylaxis of a patient suffering from said diseases.

Another aspect of the present invention is related to the use of the recombinant molecules according to the invention for the preparation of a medicament intended for the prevention and/or the treatment of the above-identified diseases or symptoms of the diseases, including the use of a nucleotide sequence encoding said recombinant molecules, and being possibly included into a vector or expressed in a cell that will be administered either in vivo or ex vivo by techniques known by one skilled in the art.

The present invention is also related to the use of variants of the molecules and proteases in order to improve the prevention and/or therapeutic method according to the invention or for reducing the possible side-effects induced by the method upon the patient. Such modification may include the deletion or the addition of one or more nucleotides, amino acids or chemical group to the molecules or nucleotide sequences encoding the molecules.

The present invention also provides a method of inhibiting HIV infectivity of a cell comprising contacting a cell with the a chemokine compound having the sequence of SEQ ID NO: 4, wherein at least 6, but not more than 15 amino acid residues are deleted from the N-terminal end, wherein cell is contacted with the chemokine compound prior to exposure of the cell to HIV.

The present invention also provides a method of inhibiting HIV infection in an animal comprising administering to an animal a pharmaceutical composition comprising chemokine compound having the sequence of SEQ ID NO: 4, wherein at least 6, but not more than 15 amino acid residues are deleted from the N-terminal end, and a pharmaceutically acceptable carrier, wherein the pharmaceutical composition is administered prior to the exposure of the animal to HIV.

The present invention will be described hereafter in reference to the enclosed figures.

### BRIEF DESCRIPTION OF THE DRAWINGS

FIG. 1 is a representation of the purification of the compound PHC-1 from human hemofiltrate (SEQ ID Nos. 7-14).

FIG. 2 shows the N-terminal sequence of the chemokine compounds of the present invention. The molecular weight indicated was measured by electrospray mass spectrometry.

FIG. 3 shows the binding (a) and functional activity (b) of chemokine compounds useful in the present invention on CCR5. Binding and activity curves are representative of at least three independent experiments. Data represent the mean and SEM for measurements performed in triplicate.

FIG. 4 shows the binding (a) and functional activity (b) of N-terminally modified chemokine compound analogs on CCR1. Binding and functional activity curves are representative of at least three independent experiments. Data represent the mean and SEM for measurements performed in triplicate.

FIG. 5 shows the binding and functional parameters of CCR1 and CCR5 for chemokine compounds useful in the present invention.

FIG. 6 shows the functional activity and binding of chemokine compounds on CCR1, CCR3, and CCR5 receptors expressed in CHO-K1 cells.

FIG. 7 shows the binding (a) and functional activity (b) of N-terminally modified chemokine compound analogs on CCR1. Binding and functional activity curves are representative of at least three independent experiments. Data represent the mean and SEM for measurements performed in triplicate.

FIG. 8 shows the binding (a) and functional activity (b) of N-terminally modified chemokine compound analogs on CCR5. Binding and functional activity curves are representative of at least three independent experiments. Data represent the mean and SEM for measurements performed in triplicate.

FIG. 9 is a representation of the calcium mobilisation and the chemotaxis induced by a PHC-1 compound in primary cell populations.

FIG. 10 is a representation of the inhibition of HIV-1 infection.

FIG. 11 is a representation of the screening of human cell lines for PHC-1 proteolytic activation.

FIG. 12 is a representation of the inhibition of proteolytic generation of PHC-1 by various serine protease inhibitors.

FIG. 13 shows the sequences of chemokines and chemokine compounds useful in the present invention.

### DETAILED DESCRIPTION OF THE INVENTION

It has been shown that specifically processed chemokines PHC-1 and PHC-2 can be isolated from human hemofiltrate, and represent highly active agonists of different chemokine receptors. The processed chemokine PHC-2 is an N-terminal truncated form of PHC-1. Both polypeptides occur in human hemofiltrate and may represent the naturally processed and biologically highly active forms of the known chemokine precursor sequence of the chemokines HCC-1 (Schulz-Knappe et al 1996, Journ. Exp. Med. 183, pp. 295-299; accession No. Q16627) or HCC-3 (accession No. Q13954).

The newly identified chemokines PHC-1 and PHC-2 belong to the family of beta-chemokines. These beta-chemokines have numerous functions related to inflammation or wound healing, immunoregulation, cancer, infectious diseases and to a number of additional disease conditions.

The peptide PHC-1 according to the invention shows a very high binding affinity and a very potent stimulatory activity for the chemokine receptors CCR1, CCR3, and CCR5. No effects of PHC-1 on the chemokine receptors, CCR2, CCR4, CCR6, CCR7, CCR8, CCR9, CXCR1 and CXCR4 has been observed. The peptide PHC-1 according to the invention affects chemotaxis/migration of eosinophils, T-lymphocytes, monocytes and dendritic cells and, may therefore be involved in inflammatory conditions in humans or may be used as a therapeutic and/or diagnostic agent for treating or diagnosing inflammatory diseases, immunological diseases, cancer diseases and infections such as viral, bacterial, fungal and protozoan infections, particularly infections caused by HIV-1 or HIV-2.

Due to the very high binding affinity of the PHC-1 polypeptide to the chemokine receptor CCR5, which represents the most important HIV-1 coreceptor, the PHC-1 polypeptide affects a very potent inhibition of HIV infection and HIV replication in human cells.

The processed chemokines PHC-1 and PHC-2 are of human origin and can be obtained by an isolation procedure departing from human blood ultrafiltrate (hemofiltrate) and by using biological assay systems to determine their biological activity. To achieve the purification of PHC-1 and PHC-2, peptides are prepared from human hemofiltrate as described recently in the literature (Schulz-Knappe et al. 1997, J. Chrom. A, 776, 125-132). The obtained hemofiltrate fractions are screened for their chemokine receptor-stimulatory activity. Then the biologically active fractions are further purified by chromatographic procedures using diverse reverse phase column chromatographic steps.

The biologically active peptides obtained by chromatographical purification are subjected to a structure determination including mass spectrometry and peptide sequence analysis.

In addition to the recombinant production of PHC-1 and PHC-2, a stepwise total synthesis on usual solid phases in terms of Merrifield synthesis is also possible.

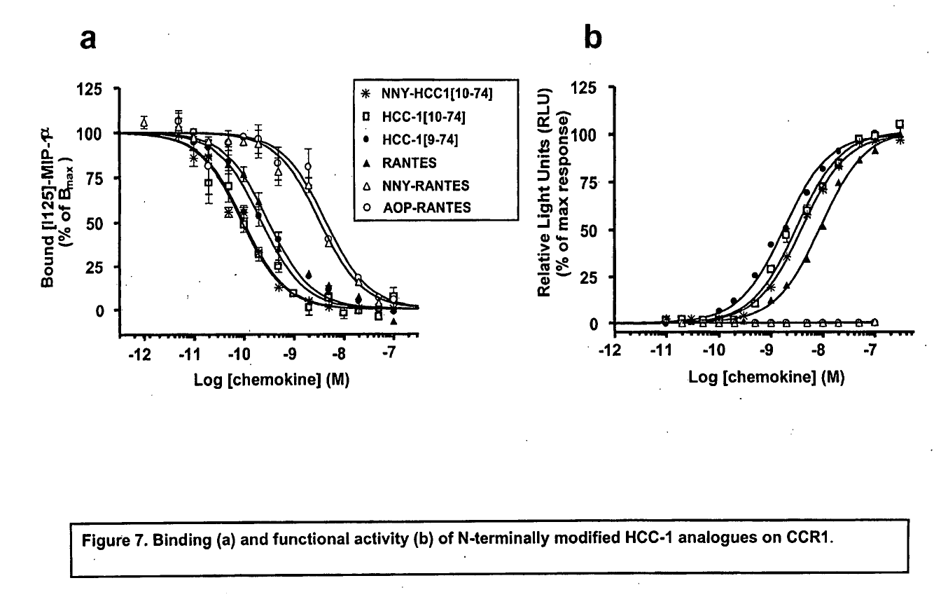

The present invention provides further, chemokine compounds derived from the HCC-1 protein (SEQ ID NO: 4). Specifically, the invention provides chemokine compounds which have the sequence of SEQ ID NO: 4, but wherein at least 6, but not more than 15 amino acid residues are deleted from the N-terminus. These chemokine compounds are shown as HCC-1[7-74], HCC-1 [8-74], HCC-1[10-74], and HCC-1[11-74] in FIG. 7, and correspond to SEQ ID Nos. 15, 16, 17, and 18, respectively. The chemokine compounds of the invention show a very high binding affinity and a very potent stimulatory activity for the chemokine receptors CCR1 and CCR5. The truncated HCC-1 compounds of the invention (i.e., “chemokine compounds”) may be generated by proteolytic processing of the HCC-1 molecule (SEQ ID NO: 4), using methods known to those of skill in the art, or may be synthesized using techniques which are well established in the art.

### Vectors and Host Cells

In one embodiment, the present invention provides both vector constructs comprising a nucleic acid sequence encoding the chemokine compounds of the present invention, and one or more host cells comprising such a vector.

A “vector” for purposes of the present invention may be any vector known to those of skill in the art such as a plasmid or viral vector, into which a nucleic acid sequence encoding a chemokine compound of the present invention has been inserted, in a forward or reverse orientation. The construct also will include regulatory sequences, including, for example, a promoter, operably linked to the sequence. Large numbers of suitable vectors and promoters are known to those of skill in the art, and are commercially available. The following vectors are provided by way of example. Bacterial: pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, pXT1, pSG (Stratagene) pSVK3, pBPV, pMSG, pSVL (Pharmacia). However, any other plasmid or vector may be used as long as it is replicable and viable in the host.

Promoter regions can be selected from any characterized gene and incorporated into appropriate vectors using techniques well known in the art. Two appropriate vectors are pKK232-8 and pCM7. Particular named bacterial promoters include lacI, lacZ, T3, T₇, gpt, PL and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

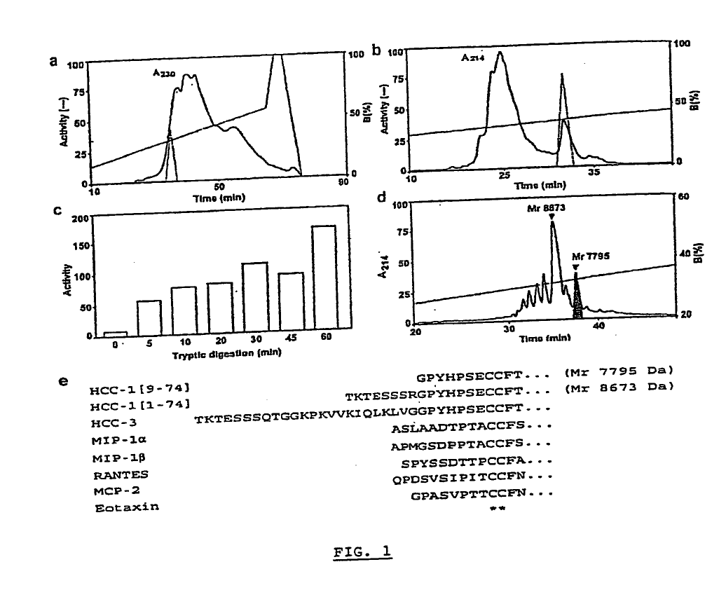

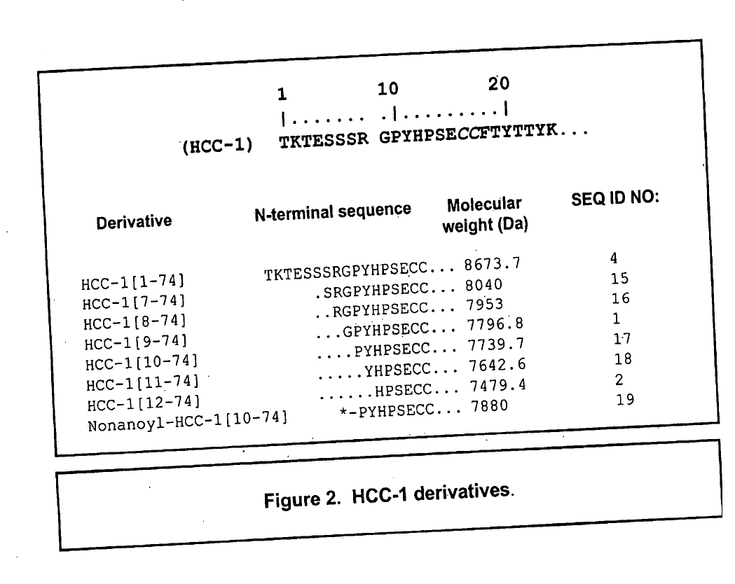

A host cell containing an above-described construct may be a higher eukaryotic cell, such as a mammalian cell, or a lower eukaryotic cell, such as a yeast cell, or the host cell may be a prokaryotic cell, such as a bacterial cell. A host cell that is useful according to the invention can be any cell into which a nucleic acid sequence encoding a receptor according to the invention can be introduced such that the receptor is expressed at natural levels or above natural levels, as defined herein. Preferably a receptor of the invention that is expressed in a cell exhibits normal or near normal pharmacology, as defined herein. Most preferably a receptor of the invention that is expressed in a cell comprises the nucleotide or amino acid sequence presented in FIG. 1 or a nucleotide or amino acid sequence that is at least 70% identical to the amino acid sequence presented in FIG. 1.

According to a preferred embodiment of the present invention, a cell is selected from the group consisting of COS-7 cells, a CHO cell, a LM (TK-) cell, a NIH-3T3 cell, HEK-293 cell, K-562 cell or a 1321N1 astrocytoma cell but also other transfectable cell lines.

Introduction of the construct into the host cell can be effected by calcium phosphate transfection, DEAE-Dextran mediated transfection, liposome mediated transfection, or electroporation (Ausubel et al., supra, 1992, pp. 9-5 to 9-14). The constructs in host cells can be used in a conventional manner to produce the gene product encoded by the recombinant sequence (i.e., a chemokine compound). Alternatively, the polypeptides of the invention can be synthetically produced by conventional peptide synthesizers.

Chemokine compounds can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, 1989, (Cold Spring Harbor Press, Cold Spring Harbor, N.Y.), the disclosure of which is hereby incorporated by reference.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Transcription of a DNA encoding the polypeptides of the present invention by higher eukaryotes is increased by inserting an enhancer sequence into the vector. Enhancers are cis-acting elements of DNA, usually about from 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers. Useful expression vectors for bacterial use are constructed by inserting a structural DNA sequence encoding a desired protein together with suitable translation initiation and termination signals in operable reading phase with a functional promoter. The vector may include one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, PKK223-3 (Pharmacia Fine Chemicals, Uppsala, Sweden) and GEM1 (Promega, Madison, Wis.). These pBR322 “backbone” sections are combined with an appropriate promoter and the structural sequence to be expressed.

Following transformation of a suitable host strain and growth of the host strain to an appropriate cell density, the selected promoter is de-repressed by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents. Such methods are well-known to those skilled in the art.

Various mammalian cell culture systems can also be employed to express recombinant protein. Examples of mammalian expression systems include the COS-7 lines of monkey kidney fibroblasts, described by Gluzman, 1981, Cell, 23:175, and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, a polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites, may be used to provide the required nontranscribed genetic elements. The expressed recombinant protein encoded by the gene sequence comprising the polynucleotide of the invention may be isolated, if necessary, by means known to those skilled in the art.

### Assays to Identify Modulators of Chemokine Activity

The discovery that the chemokine compounds of the present invention are bind and activate chemokine receptors permits screening assays to identify compounds which modulate chemokine compound binding and activation of the chemokine receptors. The screening assays will have two general approaches.

1) Ligand binding assays, in which cells expressing chemokine receptors, or membrane extracts from such cells are exposed to a labeled chemokine compound polypeptide and candidate compound. Alternatively, an analog of a chemokine compound may be used, wherein the analog comprises a moiety linked to the N-terminus of the chemokine compound. Following incubation, the reaction mixture is measured for specific binding of the chemokine compound, or analog thereof to the chemokine receptor. Compounds that interfere with or displace chemokine compounds can be agonists, antagonists or inverse agonists of chemokine receptor activity. Functional analysis can be performed on positive compounds to determine which of these categories they fit.
2) Functional assays, in which a signaling activity of a chemokine receptor is measured.
a) For agonist screening, cells expressing a chemokine receptor or membranes prepared from them are incubated with candidate compound, and a signaling activity of the chemokine receptor is measured. The assays are validated using a chemokine compound of the invention as agonist, and the activity induced by compounds that modulate receptor activity is compared to that induced by the chemokine compound. An agonist or partial agonist will have a maximal biological activity corresponding to at least 10% of the maximal activity of the chemokine compound when the agonist or partial agonist is present at 10 μM or less, and preferably will have 50%, 75%, 100% or more, including 2-fold, 5-fold, 10-fold or more activity than the chemokine compound.
b) For antagonist screening, cells expressing a chemokine receptor or membranes isolated from them are assayed for signaling activity in the presence of a chemokine compound polypeptide with or without a candidate modulator compound. Antagonists will reduce the level of chemokine compound-stimulated receptor activity by at least 10%, relative to reactions lacking the antagonist.
c) For inverse agonist screening, cells expressing a chemokine receptor or membranes isolated form such cells are assayed for constitutive receptor activity. The cell expressing the chemokine receptor or membrane fraction is then exposed to a candidate inverse agonist and receptor signaling activity is again measured. The candidate inverse agonist is confirmed as an inverse agonist if the receptor activity measured in the presence of the inverse agonist is reduced by at least 10% relative to the constitutive activity of the chemokine receptor.

### Ligand Binding and Displacement Assays:

One can use chemokine receptor polypeptides expressed on a cell, or isolated membranes containing receptor polypeptides, along with a chemokine compound polypeptide in order to screen for compounds that inhibit the binding of chemokine compounds to a chemokine receptor. When identified in an assay that measures binding or chemokine compound polypeptide displacement alone, compounds will have to be subjected to functional testing to determine whether they act as agonists, antagonists or inverse agonists.

For displacement experiments, cells expressing a chemokine receptor polypeptide (generally 25,000 cells per assay or 1 to 100 μg of membrane extracts) are incubated in binding buffer (e.g., 50 mM Hepes pH 7.4; 1 mM CaCl₂ 0.5% Bovine Serum Albumin (BSA) Fatty Acid-Free; and 0 5 mM MgCl₂) for 1.5 hrs (at, for example, 27° C.) with labeled chemokine compound polypeptide in the presence or absence of increasing concentrations of a candidate modulator. To validate and calibrate the assay, control competition reactions using increasing concentrations of unlabeled chemokine compound polypeptide can be performed. After incubation, cells are washed extensively, and bound, labeled chemokine compound is measured as appropriate for the given label (e.g., scintillation counting, enzyme assay, fluorescence, etc.). A decrease of at least 10% in the amount of labeled chemokine compound polypeptide bound in the presence of candidate modulator indicates displacement of binding by the candidate modulator. Candidate modulators are considered to bind specifically in this or other assays described herein if they displace 50% of labeled chemokine compound at a concentration of 10 μM or less (i.e., EC₅₀ is 10 μM or less).

Alternatively, binding or displacement of binding can be monitored by surface plasmon resonance (SPR). Surface plasmon resonance assays can be used as a quantitative method to measure binding between two molecules by the change in mass near an immobilized sensor caused by the binding or loss of binding of a chemokine compound polypeptide from the aqueous phase to a chemokine receptor polypeptide immobilized in a membrane on the sensor. This change in mass is measured as resonance units versus time after injection or removal of the chemokine compound polypeptide or candidate modulator and is measured using a Biacore Biosensor (Biacore AB). A chemokine receptor can be immobilized on a sensor chip (for example, research grade CM5 chip; Biacore AB) in a thin film lipid membrane according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213:219, each of which is incorporated herein by reference.). Sarrio et al. demonstrated that SPR can be used to detect ligand binding to the GPCR A(1) adenosine receptor immobilized in a lipid layer on the chip (Sarrio et al., 2000, Mol. Cell. Biol. 20: 5164-5174, incorporated herein by reference). Conditions for chemokine compound binding to a chemokine receptor in an SPR assay can be fine-tuned by one of skill in the art using the conditions reported by Sarrio et al. as a starting point.

SPR can assay for modulators of binding in at least two ways. First, a chemokine compound polypeptide can be pre-bound to immobilized chemokine receptor polypeptide, followed by injection of candidate modulator at approximately 10 μl/min flow rate and a concentration ranging from 1 nM to 100 μM, preferably about 1 μM. Displacement of the bound chemokine compound can be quantitated, permitting detection of modulator binding.

Alternatively, the membrane-bound chemokine receptor polypeptide can be pre-incubated with candidate modulator and challenged with a chemokine compound polypeptide. A difference in chemokine compound binding to the chemokine receptor exposed to modulator relative to that on a chip not pre-exposed to modulator will demonstrate binding. In either assay, a decrease of 10% or more in the amount of a chemokine compound polypeptide bound is in the presence of candidate modulator, relative to the amount of a chemokine compound polypeptide bound in the absence of candidate modulator indicates that the candidate modulator inhibits the interaction of chemokine receptor and chemokine compound.

Another method of measuring inhibition of binding of a chemokine compound polypeptide to chemokine receptor uses fluorescence resonance energy transfer (FRET). FRET is a quantum mechanical phenomenon that occurs between a fluorescence donor (D) and a fluorescence acceptor (A) in close proximity to each other (usually <100 A of separation) if the emission spectrum of D overlaps with the excitation spectrum of A. The molecules to be tested, e.g., a chemokine compound polypeptide and a chemokine receptor polypeptide, are labeled with a complementary pair of donor and acceptor fluorophores. While bound closely together by the chemokine receptor:chemokine compound interaction, the fluorescence emitted upon excitation of the donor fluorophore will have a different wavelength than that emitted in response to that excitation wavelength when the polypeptides are not bound, providing for quantitation of bound versus unbound polypeptides by measurement of emission intensity at each wavelength. Donor:Acceptor pairs of fluorophores with which to label the polypeptides are well known in the art. Of particular interest are variants of the A. victoria GFP known as Cyan FP (CFP, Donor(D)) and Yellow FP (YFP, Acceptor(A)). The GFP variants can be made as fusion proteins with the respective members of the binding pair to serve as D-A pairs in a FRET scheme to measure protein-protein interaction. Vectors for the expression of GFP variants as fusions are known in the art. As an example, a CFP-chemokine compound fusion and a YFP-chemokine receptor fusion can be made. The addition of a candidate modulator to the mixture of labeled chemokine compound and chemokine receptor proteins will result in an inhibition of energy transfer evidenced by, for example, a decrease in YFP fluorescence relative to a sample without the candidate modulator. In an assay using FRET for the detection of chemokine receptor:chemokine compound interaction, a 10% or greater decrease in the intensity of fluorescent emission at the acceptor wavelength in samples containing a candidate modulator, relative to samples without the candidate modulator, indicates that the candidate modulator inhibits chemokine receptor:chemokine compound interaction.

A variation on FRET uses fluorescence quenching to monitor molecular interactions. One molecule in the interacting pair can be labeled with a fluorophore, and the other with a molecule that quenches the fluorescence of the fluorophore when brought into close apposition with it. A change in fluorescence upon excitation is indicative of a change in the association of the molecules tagged with the fluorophore:quencher pair. Generally, an increase in fluorescence of the labeled chemokine receptor polypeptide is indicative that the chemokine compound polypeptide bearing the quencher has been displaced. For quenching assays, a 10% or greater increase in the intensity of fluorescent emission in samples containing a candidate modulator, relative to samples without the candidate modulator, indicates that the candidate modulator inhibits chemokine receptor:chemokine compound interaction.

In addition to the surface plasmon resonance and FRET methods, fluorescence polarization measurement is useful to quantitate protein-protein binding. The fluorescence polarization value for a fluorescently-tagged molecule depends on the rotational correlation time or tumbling rate. Protein complexes, such as those formed by chemokine receptor associating with a fluorescently labeled chemokine compound polypeptide, have higher polarization values than uncomplexed, labeled chemokine compound. The inclusion of a candidate inhibitor of the chemokine receptor:chemokine compound interaction results in a decrease in fluorescence polarization, relative to a mixture without the candidate inhibitor, if the candidate inhibitor disrupts or inhibits the interaction of chemokine receptor with chemokine compound. Fluorescence polarization is well suited for the identification of small molecules that disrupt the formation of polypeptide or protein complexes. A decrease of 10% or more in fluorescence polarization in samples containing a candidate modulator, relative to fluorescence polarization in a sample lacking the candidate modulator, indicates that the candidate modulator inhibits chemokine receptor:chemokine compound interaction.

Another alternative for monitoring chemokine receptor:chemokine compound interactions uses a biosensor assay. ICS biosensors have been described by AMBRI (Australian Membrane Biotechnology Research Institute). In this technology, the association of macromolecules such as chemokine receptor and chemokine compound, is coupled to the closing of gramicidin-facilitated ion channels in suspended membrane bilayers and thus to a measurable change in the admittance (similar to impedance) of the biosensor. This approach is linear over six orders of magnitude of admittance change and is ideally suited for large scale, high throughput screening of small molecule combinatorial libraries. A 10% or greater change (increase or decrease) in admittance in a sample containing a candidate modulator, relative to the admittance of a sample lacking the candidate modulator, indicates that the candidate modulator inhibits the interaction of chemokine receptor and chemokine compound.

It is important to note that in assays of protein-protein interaction, it is possible that a modulator of the interaction need not necessarily interact directly with the domain(s) of the proteins that physically interact. It is also possible that a modulator will interact at a location removed from the site of protein-protein interaction and cause, for example, a conformational change in the chemokine receptor polypeptide. Modulators (inhibitors or agonists) that act in this manner are nonetheless of interest as agents to modulate the activity of chemokine receptor.

It should be understood that any of the binding assays described herein can be performed with a non-chemokine compound ligand (for example, agonist, antagonist, etc.) of chemokine receptor, e.g., a small molecule identified as described herein. In practice, the use of a small molecule ligand or other non-chemokine compound ligand has the benefit that non-polypeptide chemical compounds are generally less expensive and easier to produce in purified form than polypeptides such as chemokine compound. Thus, a non-chemokine compound ligand is better suited to high-throughput assays for the identification of agonists, antagonists or inverse agonists than full length chemokine compound. This advantage in no way erodes the importance of assays using chemokine compound, however, as such assays are well suited for the initial identification of non-chemokine compound ligands.

Any of the binding assays described can be used to determine the presence of an agent in a sample, e.g., a tissue sample, that binds to the chemokine receptor molecule, or that affects the binding of chemokine compound to the receptor. To do so, chemokine receptor polypeptide is reacted with chemokine compound polypeptide or another ligand in the presence or absence of the sample, and chemokine compound or ligand binding is measured as appropriate for the binding assay being used. A decrease of 10% or more in the binding of chemokine compound or other ligand indicates that the sample contains an agent that modulates chemokine compound or ligand binding to the receptor polypeptide.

### Functional Assays of Receptor Activity

i. GTPase/GTP Binding Assays:

For chemokine receptors, a measure of receptor activity is the binding of GTP by cell membranes containing receptors. In the method described by Traynor and Nahorski, 1995, Mol. Pharmacol. 47: 848-854, incorporated herein by reference, one essentially measures G-protein coupling to membranes by measuring the binding of labeled GTP. For GTP binding assays, membranes isolated from cells expressing the receptor are incubated in a buffer containing 20 mM HEPES, pH 7.4, 100 mM NaCl, and 10 mM MgCl2, 80 pM³⁵S-GTPγS and 3 μM GDP. The assay mixture is incubated for 60 minutes at 30° C., after which unbound labeled GTP is removed by filtration onto GF/B filters. Bound, labeled GTP is measured by liquid scintillation counting. In order to assay for modulation of chemokine compound-induced chemokine receptor activity, membranes prepared from cells expressing a chemokine receptor polypeptide are mixed with a chemokine compound polypeptide, and the GTP binding assay is performed in the presence and absence of a candidate modulator of chemokine receptor activity. A decrease of 10% or more in labeled GTP binding as measured by scintillation counting in an assay of this kind containing candidate modulator, relative to an assay without the modulator, indicates that the candidate modulator inhibits chemokine receptor activity.

A similar GTP-binding assay can be performed without a chemokine compound to identify compounds that act as agonists. In this case, chemokine compound-stimulated GTP binding is used as a standard. A compound is considered an agonist if it induces at least 50% of the level of GTP binding induced by the chemokine compound when the compound is present at 1 μM or less, and preferably will induce a level the same as or higher than that induced by chemokine compound.

GTPase activity is measured by incubating the membranes containing a chemokine receptor polypeptide with γ³²P-GTP. Active GTPase will release the label as inorganic phosphate, which is detected by separation of free inorganic phosphate in a 5% suspension of activated charcoal in 20 mM H₃PO₄, followed by scintillation counting. Controls include assays using membranes isolated from cells not expressing chemokine receptor (mock-transfected), in order to exclude possible non-specific effects of the candidate compound.

In order to assay for the effect of a candidate modulator on chemokine receptor-regulated GTPase activity, membrane samples are incubated with a chemokine compound polypeptide, with and without the modulator, followed by the GTPase assay. A change (increase or decrease) of 10% or more in the level of GTP binding or GTPase activity relative to samples without modulator is indicative of chemokine receptor modulation by the candidate modulator.

ii. Downstream Pathway Activation Assays:

a. Calcium flux—The Aequorin-based Assay.

The aequorin assay takes advantage of the responsiveness of mitochondrial apoaequorin to intracellular calcium release induced by the activation of GPCRs (Stables et al., 1997, Anal. Biochem. 252:115-126; Detheux et al, 2000, J. Exp. Med., 192 1501-1508; both of which are incorporated herein by reference). Briefly, chemokine receptor-expressing clones are transfected to coexpress mitochondrial apoaequorin and Gα16. Cells are incubated with 5 μM Coelenterazine H (Molecular Probes) for 4 hours at room temperature, washed in DMEM-F12 culture medium and resuspended at a concentration of 0.5×10⁶ cells/ml. Cells are then mixed with test agonist peptides and light emission by the aequorin is recorded with a luminometer for 30 sec. Results are expressed as Relative Light Units (RLU). Controls include assays using membranes isolated from cells not expressing the 3chemokine receptor (mock-transfected), in order to exclude possible non-specific effects of the candidate compound.

Aequorin activity or intracellular calcium levels are “changed” if light intensity increases or decreases by 10% or more in a sample of cells, expressing a chemokine receptor polypeptide and treated with a candidate modulator, relative to a sample of cells expressing the chemokine receptor polypeptide but not treated with the candidate modulator or relative to a sample of cells not expressing the chemokine receptor polypeptide (mock-transfected cells) but treated with the candidate modulator.

When performed in the absence of a chemokine compound polypeptide, the assay can be used to identify an agonist of chemokine receptor activity. When the assay is performed in the presence of a chemokine compound polypeptide, it can be used to assay for an antagonist.

b. Adenylate Cyclase Assay:

Assays for adenylate cyclase activity are described by Kenimer & Nirenberg, 1981, Mol. Pharmacol. 20: 585-591, incorporated herein by reference. That assay is a modification of the assay taught by Solomon et al., 1974, Anal. Biochem. 58: 541-548, also incorporated herein by reference. Briefly, 100 μl reactions contain 50 mM Tris-HCl (pH 7.5), 5 mM MgCl₂, 20 mM creatine phosphate (disodium salt), 10 units (71 μg of protein) of creatine phosphokinase, 1 mM α-³²P-ATP (tetrasodium salt, 2 μCi), 0.5 mM cyclic AMP, G-³H-labeled cyclic AMP (approximately 10,000 cpm), 0.5 mM Ro20-17724, 0.25% ethanol, and 50-200 μg of protein homogenate to be tested (i.e., homogenate from cells expressing or not expressing a chemokine receptor polypeptide, treated or not treated with a chemokine compound polypeptide with or without a candidate modualtor). Reaction mixtures are generally incubated at 37° C. for 6 minutes. Following incubation, reaction mixtures are deproteinized by the addition of 0.9 ml of cold 6% trichloroacetic acid. Tubes are centrifuged at 1800×g for 20 minutes and each supernatant solution is added to a Dowex AG50W-X4 column. The cAMP fraction from the column is eluted with 4 ml of 0.1 mM imidazole-HCl (pH 7.5) into a counting vial. Assays should be performed in triplicate. Control reactions should also be performed using protein homogenate from cells that do not express a chemokine receptor polypeptide.

According to the invention, adenylate cyclase activity is “changed” if it increases or decreases by 10% or more in a sample taken from cells treated with a candidate modulator of chemokine receptor activity, relative to a similar sample of cells not treated with the candidate modulator or relative to a sample of cells not expressing the chemokine receptor polypeptide (mock-transfected cells) but treated with the candidate modulator.

c. cAMP Assay:

Intracellular or extracellular cAMP is measured using a cAMP radioimmunoassay (RIA) or cAMP binding protein according to methods widely known in the art. For example, Horton & Baxendale, 1995, Methods Mol. Biol. 41: 91-105, which is incorporated herein by reference, describes an RIA for cAMP.

A number of kits for the measurement of cAMP are commercially available, such as the High Efficiency Fluorescence Polarization-based homogeneous assay marketed by LJL Biosystems and NEN Life Science Products. Control reactions should be performed using extracts of mock-transfected cells to exclude possible non-specific effects of some candidate modulators.

The level of cAMP is “changed” if the level of cAMP detected in cells, expressing a chemokine receptor polypeptide and treated with a candidate modulator of chemokine receptor activity (or in extracts of such cells), using the RIA-based assay of Horton & Baxendale, 1995, supra, increases or decreases by at least 10% relative to the cAMP level in similar cells not treated with the candidate modulator.

d. Phospholipid Breakdown, DAG Production and Inositol Triphosphate Levels:

Receptors that activate the breakdown of phospholipids can be monitored for changes due to the activity of known or suspected modulators of chemokine receptor by monitoring phospholipid breakdown, and the resulting production of second messengers DAG and/or inositol triphosphate (IP₃). Methods of measuring each of these are described in Phospholipid Signaling Protocols, edited by Ian M. Bird. Totowa, N.J., Humana Press, 1998, which is incorporated herein by reference. See also Rudolph et al., 1999, J. Biol. Chem. 274: 11824-11831, incorporated herein by reference, which also describes an assay for phosphatidylinositol breakdown. Assays should be performed using cells or extracts of cells expressing chemokine receptor, treated or not treated with a chemokine compound polypeptide with or without a candidate modulator. Control reactions should be performed using mock-transfected cells, or extracts from them in order to exclude possible non-specific effects of some candidate modulators.

According to the invention, phosphatidylinositol breakdown, and diacylglycerol and/or inositol triphosphate levels are “changed” if they increase or decrease by at least 10% in a sample from cells expressing a chemokine receptor polypeptide and treated with a candidate modulator, relative to the level observed in a sample from cells expressing a chemokine receptor polypeptide that is not treated with the candidate modulator.

e. PKC Activation Assays:

Growth factor receptor tyrosine kinases tend to signal via a pathway involving activation of Protein Kinase C (PKC), which is a family of phospholipid- and calcium-activated protein kinases. PKC activation ultimately results in the transcription of an array of proto-oncogene transcription factor-encoding genes, including c-fos, c-myc and c-jun, proteases, protease inhibitors, including collagenase type I and plasminogen activator inhibitor, and adhesion molecules, including intracellular adhesion molecule I (ICAM I). Assays designed to detect increases in gene products induced by PKC can be used to monitor PKC activation and thereby receptor activity. In addition, the activity of receptors that signal via PKC can be monitored through the use of reporter gene constructs driven by the control sequences of genes activated by PKC activation. This type of reporter gene-based assay is discussed in more detail below.

For a more direct measure of PKC activity, the method of Kikkawa et al., 1982, J. Biol. Chem. 257: 13341, incorporated herein by reference, can be used. This assay measures phosphorylation of a PKC substrate peptide, which is subsequently separated by binding to phosphocellulose paper. This PKC assay system can be used to measure activity of purified kinase, or the activity in crude cellular extracts. Protein kinase C sample can be diluted in 20 mM HEPES/2 mM DTT immediately prior to assay.

The substrate for the assay is the peptide Ac-FKKSFKL-NH2, derived from the myristoylated alanine-rich protein kinase C substrate protein (MARCKS). The Kₘ of the enzyme for this peptide is approximately 50 μM. Other basic, protein kinase C-selective peptides known in the art can also be used, at a concentration of at least 2-3 times their Kₘ. Cofactors required for the assay include calcium, magnesium, ATP, phosphatidylserine and diacylglycerol. Depending upon the intent of the user, the assay can be performed to determine the amount of PKC present (activating conditions) or the amount of active PCK present (non-activating conditions). For most purposes according to the invention, non-activating conditions will be used, such that the PKC that is active in the sample when it is isolated is measured, rather than measuring the PKC that can be activated. For non-activating conditions, calcium is omitted in the assay in favor of EGTA.

The assay is performed in a mixture containing 20 mM HEPES, pH 7.4, 1-2 mM DTT, 5 mM MgCl₂, 100 μM ATP, ˜1 μCi γ-³²P-ATP, 100 μg/ml peptide substrate (˜100 μM), 140 μM/3.8 μM phosphatidylserine/diacylglycerol membranes, and 100 μM calcium (or 500 μM EGTA). 48 μl of sample, diluted in 20 mM HEPES, pH 7.4, 2 mM DTT is used in a final reaction volume of 80 μl. Reactions are performed at 30° C. for 5-10 minutes, followed by addition of 25 μl of 100 mM ATP, 100 mM EDTA, pH 8.0, which stops the reactions.

After the reaction is stopped, a portion (85 μl) of each reaction is spotted onto a Whatman P81 cellulose phosphate filter, followed by washes: four times 500 ml in 0.4% phosphoric acid, (5-10 min per wash); and a final wash in 500 ml 95% EtOH, for 2-5 mm. Bound radioactivity is measured by scintillation counting. Specific activity (cpm/nmol) of the labeled ATP is determined by spotting a sample of the reaction onto P81 paper and counting without washing. Units of PKC activity, defined as nmol phosphate transferred per mm, are calculated as follows:
The activity, in UNITS (nmol/min) is:

$$= \frac{( cpm on paper ) \times ( 105 μ l total / 85 μ l spotted )}{( assay time , min ) ( specific activity of ATP cpm / nmol ) .}$$

An alternative assay can be performed using a Protein Kinase C Assay Kit sold by Pan Vera (Cat. #P2747).

Assays are performed on extracts from cells expressing a chemokine receptor polypeptide, treated or not treated with a chemokine compound polypeptide with or without a candidate modulator. Control reactions should be performed using mock-transfected cells, or extracts from them in order to exclude possible non-specific effects of some candidate modulators.

According to the invention, PKC activity is “changed” by a candidate modulator when the units of PKC measured by either assay described above increase or decrease by at least 10%, in extracts from cells expressing chemokine receptor and treated with a candidate modulator, relative to a reaction performed on a similar sample from cells not treated with a candidate modulator.

f. Kinase Assays:

MAP kinase activity can be assayed using any of several kits available commercially, for example, the p38 MAP Kinase assay kit sold by New England Biolabs (Cat # 9820) or the FlashPlate™ MAP Kinase assays sold by Perkin-Elmer Life Sciences.

MAP Kinase activity is “changed” if the level of activity is increased or decreased by 10% or more in a sample from cells, expressing a chemokine receptor polypeptide, treated with a candidate modulator relative to MAP kinase activity in a sample from similar cells not treated with the candidate modulator.

Direct assays for tyrosine kinase activity using known synthetic or natural tyrosine kinase substrates and labeled phosphate are well known, as are similar assays for other types of kinases (e.g., Ser/Thr kinases). Kinase assays can be performed with both purified kinases and crude extracts prepared from cells expressing a chemokine receptor polypeptide, treated with or without a chemokine compound polypeptide, with or without a candidate modulator. Control reactions should be performed using mock-transfected cells, or extracts from them in order to exclude possible non-specific effects of some candidate modulators. Substrates can be either full length protein or synthetic peptides representing the substrate. Pinna & Ruzzene (1996, Biochem. Biophys. Acta 1314: 191-225, incorporated herein by reference) list a number of phosphorylation substrate sites useful for measuring kinase activities. A number of kinase substrate peptides are commercially available. One that is particularly useful is the “Src-related peptide,” (available from Sigma # A7433), which is a substrate for many receptor and nonreceptor tyrosine kinases. Because the assay described below requires binding of peptide substrates to filters, the peptide substrates should have a net positive charge to facilitate binding. Generally, peptide substrates should have at least 2 basic residues and a free amino terminus. Reactions generally use a peptide concentration of 0.7-1.5 mM.

Assays are generally carried out in a 25 μl volume comprising 5 μl of 5× kinase buffer (5 mg/mL BSA, 150 mM Tris-Cl (pH 7.5), 100 mM MgCl₂ depending upon the exact kinase assayed for, MnCl₂ can be used in place of or in addition to the MgCl₂), 5 μl of 1.0 mM ATP (0.2 mM final concentration), γ-³²P-ATP (100-500 cpm/pmol), 3 μl of 10 mM peptide substrate (1.2 mM final concentration), cell extract containing kinase to be tested (cell extracts used for kinase assays should contain a phosphatase inhibitor (e.g. 0.1-1 mM sodium orthovanadate)), and H₂O to 25 μl. Reactions are performed at 30° C., and are initiated by the addition of the cell extract.

Kinase reactions are performed for 30 seconds to about 30 minutes, followed by the addition of 45 μl of ice-cold 10% trichloroacetic acid (TCA). Samples are spun for 2 minutes in a microcentrifuge, and 35 μl of the supernatant is spotted onto Whatman P81 cellulose phosphate filter circles. The filters are washed three times with 500 ml cold 0.5% phosphoric acid, followed by one wash with 200 ml of acetone at room temperature for 5 minutes. Filters are dried and incorporated 32P is measured by scintillation counting. The specific activity of ATP in the kinase reaction (e.g., in cpm/pmol) is determined by spotting a small sample (2-5 μl) of the reaction onto a P81 filter circle and counting directly, without washing. Counts per minute obtained in the kinase reaction (minus blank) are then divided by the specific activity to determine the moles of phosphate transferred in the reaction.

Tyrosine kinase activity is “changed” if the level of kinase activity is increased or decreased by 10% or more in a sample from cells, expressing a chemokine receptor polypeptide, treated with a candidate modulator relative to kinase activity in a sample from similar cells not treated with the candidate modulator.

g. Transcriptional Reporters for Downstream Pathway Activation:

The intracellular signal initiated by binding of an agonist to a receptor, e.g., chemokine receptor, sets in motion a cascade of intracellular events, the ultimate consequence of which is a rapid and detectable change in the transcription or translation of one or more genes. The activity of the receptor can therefore be monitored by measuring the expression of a reporter gene driven by control sequences responsive to chemokine receptor activation.

As used herein “promoter” refers to the transcriptional control elements necessary for receptor-mediated regulation of gene expression, including not only the basal promoter, but also any enhancers or transcription-factor binding sites necessary for receptor-regulated expression. By selecting promoters that are responsive to the intracellular signals resulting from agonist binding, and operatively linking the selected promoters to reporter genes whose transcription, translation or ultimate activity is readily detectable and measurable, the transcription based reporter assay provides a rapid indication of whether a given receptor is activated.

Reporter genes such as luciferase, CAT, GFP, β-lactamase or β-galactosidase are well known in the art, as are assays for the detection of their products.

Genes particularly well suited for monitoring receptor activity are the “immediate early” genes, which are rapidly induced, generally within minutes of contact between the receptor and the effector protein or ligand. The induction of immediate early gene transcription does not require the synthesis of new regulatory proteins. In addition to rapid responsiveness to ligand binding, characteristics of preferred genes useful to make reporter constructs include: low or undetectable expression in quiescent cells; induction that is transient and independent of new protein synthesis; subsequent shut-off of transcription requires new protein synthesis; and mRNAs transcribed from these genes have a short half-life. It is preferred, but not necessary that a transcriptional control element have all of these properties for it to be useful.

An example of a gene that is responsive to a number of different stimuli is the c-fos proto-oncogene. The c-fos gene is activated in a protein-synthesis-independent manner by growth factors, hormones, differentiation-specific agents, stress, and other known inducers of cell surface proteins. The induction of c-fos expression is extremely rapid, often occurring within minutes of receptor stimulation. This characteristic makes the c-fos regulatory regions particularly attractive for use as a reporter of receptor activation.

The c-fos regulatory elements include (see, Verma et al., 1987, Cell 51: 513-514): a TATA box that is required for transcription initiation; two upstream elements for basal transcription, and an enhancer, which includes an element with dyad symmetry and which is required for induction by TPA, serum, EGF, and PMA.

The 20 bp c-fos transcriptional enhancer element located between −317 and −298 bp upstream from the c-fos mRNA cap site, is essential for serum induction in serum starved NIH 3T3 cells.

One of the two upstream elements is located at −63 to −57 and it resembles the consensus sequence for cAMP regulation.

The transcription factor CREB (cyclic AMP responsive element binding protein) is, as the name implies, responsive to levels of intracellular cAMP. Therefore, the activation of a receptor that signals via modulation of cAMP levels can be monitored by measuring either the binding of the transcription factor, or the expression of a reporter gene linked to a CREB-binding element (termed the CRE, or cAMP response element). Reporter constructs responsive to CREB binding activity are described in U.S. Pat. No. 5,919,649.

Other promoters and transcriptional control elements, in addition to the c-fos elements and CREB-responsive constructs, include the vasoactive intestinal peptide (VIP) gene promoter (cAMP responsive; Fink et al, 1988, Proc. Natl. Acad. Sci. 85:6662-6666); the somatostatin gene promoter (cAMP responsive; Montminy et al., 1986, Proc. Natl. Acad. Sci. 8.3:6682-6686); the proenkephalin promoter (responsive to cAMP, nicotinic agonists, and phorbol esters; Comb et al., 1986, Nature 323:353-356); the phosphoenolpyruvate carboxy-kinase (PEPCK) gene promoter (cAMP responsive; Short et al., 1986, J. Biol. Chem. 261:9721-9726).

Additional examples of transcriptional control elements that are responsive to changes in chemokine receptor activity include, but are not limited to those responsive to the AP-1 transcription factor and those responsive to NF-κB activity. The consensus AP-1 binding site is the palindrome TGA(C/G)TCA (Lee et al., 1987, Nature 325: 368-372; Lee et al., 1987, Cell 49: 741-752). The AP-1 site is also responsible for mediating induction by tumor promoters such as the phorbol ester 12-O-tetradecanoylphorbo-β-acetate (TPA), and are therefore sometimes also referred to as a TRE, for TPA-response element. AP-1 activates numerous genes that are involved in the early response of cells to growth stimuli. Examples of AP-1-responsive genes include, but are not limited to the genes for Fos and Jun (which proteins themselves make up AP1 activity), Fos-related antigens (Fra) 1 and 2, IκBα, ornithine decarboxylase, and annexins I and II.

A large number of genes have been identified as NF-κB responsive, and their control elements can be linked to a reporter gene to monitor GPCR activity. A small sample of the genes responsive to NF-κB includes those encoding IL-1β (Hiscott et al., 1993, Mol. Cell. Biol. 13: 6231-6240), TNF-α (Shakhov et al., 1990, J. Exp. Med. 171: 35-47), CCR5 (Liu et al., 1998, AIDS Res. Hum. Retroviruses 14: 1509-1519), P-selectin (Pan & McEver, 1995, J. Biol. Chem. 270: 23077-23083), Fas ligand (Matsui et al., 1998, J. Immunol. 161: 3469-3473), GM-CSF (Schreck & Baeuerle, 1990, Mol. Cell. Biol. 10: 1281-1286) and IκBα (Haskill et al., 1991, Cell 65: 1281-1289). Each of these references is incorporated herein by reference. Vectors encoding NF-κB-responsive reporters are also known in the art or can be readily made by one of skill in the art using, for example, synthetic NF-κB elements and a minimal promoter, or using the NF-κB-responsive sequences of a gene known to be subject to NF-κB regulation. Further, NF-κB responsive reporter constructs are commercially available from, for example, CLONTECH.

A given promoter construct should be tested by exposing chemokine receptor-expressing cells, transfected with the construct, to a chemokine compound polypeptide. An increase of at least two-fold in the expression of reporter in response to chemokine compound polypeptide indicates that the reporter is an indicator of chemokine receptor activity.

In order to assay chemokine receptor activity with a chemokine compound-responsive transcriptional reporter construct, cells that stably express a chemokine receptor polypeptide are stably transfected with the reporter construct. To screen for agonists, the cells are left untreated, exposed to candidate modulators, or exposed to a chemokine compound polypeptide, and expression of the reporter is measured. The chemokine compound-treated cultures serve as a standard for the level of transcription induced by a known agonist. An increase of at least 50% in reporter expression in the presence of a candidate modulator indicates that the candidate is a modulator of chemokine receptor activity. An agonist will induce at least as much, and preferably the same amount or more, reporter expression than the chemokine compound polypeptide. This approach can also be used to screen for inverse agonists where cells express a chemokine receptor polypeptide at levels such that there is an elevated basal activity of the reporter in the absence of chemokine compound or another agonist. A decrease in reporter activity of 10% or more in the presence of a candidate modulator, relative to its absence, indicates that the compound is an inverse agonist.

To screen for antagonists, the cells expressing chemokine receptor and carrying the reporter construct are exposed to a chemokine compound polypeptide (or another agonist) in the presence and absence of candidate modulator. A decrease of 10% or more in reporter expression in the presence of candidate modulator, relative to the absence of the candidate modulator, indicates that the candidate is a modulator of chemokine receptor activity.

Controls for transcription assays include cells not expressing chemokine receptor but carrying the reporter construct, as well as cells with a promoterless reporter construct. Compounds that are identified as modulators of chemokine receptor-regulated transcription should also be analyzed to determine whether they affect transcription driven by other regulatory sequences and by other receptors, in order to determine the specificity and spectrum of their activity.

The transcriptional reporter assay, and most cell-based assays, are well suited for screening expression libraries for proteins for those that modulate chemokine receptor activity. The libraries can be, for example, cDNA libraries from natural sources, e.g., plants, animals, bacteria, etc., or they can be libraries expressing randomly or systematically mutated variants of one or more polypeptides. Genomic libraries in viral vectors can also be used to express the mRNA content of one cell or tissue, in the different libraries used for screening of chemokine receptor.

Any of the assays of receptor activity, including the GTP-binding, GTPase, adenylate cyclase, cAMP, phospholipid-breakdown, diacylglyceorl, inositol triphosphate, PKC, kinase and transcriptional reporter assays, can be used to determine the presence of an agent in a sample, e.g., a tissue sample, that affects the activity of the chemokine receptor molecule. To do so, chemokine receptor polypeptide is assayed for activity in the presence and absence of the sample or an extract of the sample. An increase in chemokine receptor activity in the presence of the sample or extract relative to the absence of the sample indicates that the sample contains an agonist of the receptor activity. A decrease in receptor activity in the presence of chemokine compound or another agonist and the sample, relative to receptor activity in the presence of chemokine compound polypeptide alone, indicates that the sample contains an antagonist of chemokine receptor activity. If desired, samples can then be fractionated and further tested to isolate or purify the agonist or antagonist. The amount of increase or decrease in measured activity necessary for a sample to be said to contain a modulator depends upon the type of assay used. Generally, a 10% or greater change (increase or decrease) relative to an assay performed in the absence of a sample indicates the presence of a modulator in the sample. One exception is the transcriptional reporter assay, in which at least a two-fold increase or 10% decrease in signal is necessary for a sample to be said to contain a modulator. It is preferred that an agonist stimulates at least 50%, and preferably 75% or 100% or more, e.g., 2-fold, 5-fold, 10-fold or greater receptor activation than a chemokine compound.

Other functional assays include, for example, microphysiometer or biosensor assays (see Hafner, 2000, Biosens. Bioelectron. 15: 149-158, incorporated herein by reference).

### Dosage and Mode of Administration:

By way of example, a patient in need of a compound, analog, inhibitor, agonist, antagonist, polynucleotide agent or cell of the invention can be treated as follows. Cells of the invention can be administered to the patient, preferably in a biologically compatible solution or a pharmaceutically acceptable delivery vehicle, by ingestion, injection, inhalation or any number of other methods. A preferred method is endoscopic retrograde injection. The dosages administered will vary from patient to patient; a “therapeutically effective dose” can be determined, for example but not limited to, by the level of enhancement of function. Monitoring levels of introduction, the level of expression of certain genes affected by such transfer, and/or the presence or levels of the encoded product will also enable one skilled in the art to select and adjust the dosages administered. Generally, a composition including a cell of the invention will be administered in a single dose in the range of 10⁵-10⁸ cells per kg body weight, preferably in the range of 10⁶-10⁷ cells per kg body weight. This dosage may be repeated daily, weekly, monthly, yearly, or as considered appropriate by the treating physician. The invention provides that cell populations can also be removed from the patient or otherwise provided, expanded ex vivo, transduced with a plasmid containing a therapeutic gene if desired, and then reintroduced into the patient.

### Pharmaceutical Compositions:

The invention provides for compositions comprising a compound, analog, inhibitor, agonist, antagonist, polynucleotide agent or cell according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through a combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. . . . Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a Ph range of 4.5 to 5.5 that is combined with buffer prior to use.

After pharmaceutical compositions comprising a compound of the invention formulated in an acceptable carrier have been prepared, they can be placed in an appropriate container and labeled for treatment of an indicated condition with information including amount, frequency and method of administration.

The above disclosure generally describes the present invention. A more complete understanding can be obtained by reference to the following specific examples, which are provided herein for purposes of illustration only and are not intended to limit the scope of the invention.

### EXAMPLES

### Example 1

### Isolation of Hum an Chemokines PHC-1 and PHC-2 from Hemofiltrate

Peptides of human hemofiltrate were prepared as described recently in the literature (Schulz-Knappe, P. et al. 1997, J. Chrom. A, 776, 125-132). In brief, the peptides were extracted from 10,000 L of human hemofiltrate obtained from a local neurological center. The collected hemofiltrate had been derived from 40 adult patients with chronic renal disease. Immediately after blood filtration using ultrafilters with a specified cut-off of 20 kDa, the filtrate was routinely chilled to 4° C. and adjusted to pH 3 to prevent bacterial growth and proteolysis. After dilution with deionized water to a conductivity of <8 mS/cm, the batches of 800-1000 L hemofiltrate were conditioned exactly to pH 2.7 using hydrochloric acid. These batches were applied to a strong cation-exchanger (2 1 Fractogel TSK SP 650(M), Merck) and the peptides were batch-eluted with 10 1 0.5 M ammonium acetate, pH 7.0. These eluates were stored at −20° C. To eliminate remaining amounts of plasma albumine in the concentrated hemofiltrate an additional ultrafiltration step was carried out with pooled eluates corresponding to 10.000 L equivalent of hemofiltrate. Ultrafiltration was performed by a sartocon-mini (0.1 m², ps-membrane) ultrafilter with a specified Mr cut-off of 30 kDa. Filtration was driven by a transmembranous pressure gradient of 1 bar at a temperature of 5° C. and a flow rate of 5-6 L/h.

For the first purification step of PHC-1 and PHC-2 the ultrafiltrate was diluted with deionized water to a conductivity of 6.7 mS/cm, adjusted to pH 2.7 with HCl, and applied to a second 10 1 Fractogel cation-exchange column. The column was washed with 0.01 M HCl until conductivity was below 1 mS/cm. Stepwise batch elution of the bound peptides was performed using the eight following buffers: I: 0.1 N citric acid pH 3.6; II: 0.1 N acetic acid+0.1 M sodium acetate, pH 4.5; III: 0.1 M malic acid, pH 5.0; IV: 0.1 succinic acid, pH 5.6; V: 0.1 M sodium dihydrogenphosphate, pH 6.6; VI: 0.1 M disodium hydrogenphosphate, pH 7.4; VII: 0.1 M ammonium carbonate, pH 9.0; VIII: water, pH 7.0 (desalting step). The resulting pH pool fractions (15 to 25 L) were collected, acidified to pH 2-3, and immediately subjected to the second separation step.

For the second purification step of PHC-1 and PHC-2 the eluate of pH pool fraction No. 5 was loaded onto a Source RPC column (15 μm, 10×12.5 cm, Pharmacia), washed with two column volumes of solvent A (10 mM HCl) and separation was performed at a flow rate of 200 ml/mm by a 8 1 gradient from 100% A (water, 10 mM HCl) to 60% B (80% acetonitrile (v/v), 10 mM HCl). Fractions of 200 ml were collected and the absorbance at 280 nm was monitored. Aliquots of these peptide fractions were lyophilized and tested for bioactivity as described below.

Fraction 21 contained the biologically active peptides according to this invention (FIG. 1A).

For the third purification step of PHC-1 and PHC-2 the bioactive fraction 21 was loaded on a RP-C18 column (15-20 μm, 300 A, 47×300 mm; Vydac, Hesperia, USA) and separation was performed under a flow rate of 40 ml/min using the following gradient and buffers: from 90% A (water, 10 mM HCl) to 50% B (80% acetonitrile, 10 mM HCl) in 48 min. Single fractions of 1 min were collected, monitoring the absorbance at 214 nm, and tested for bioactivity as described below. Fraction 12 contained the biologically active peptides according to this invention.

For the fourth purification step of PHC-1 and PHC-2 the bioactive fraction 12 was loaded on a RP-C4 column (5 μm, 100 A, 20×250 mm; Biotek Silica, Östringen, Germany) and separation was performed under a flow rate of 7 ml/min using the following gradient and buffers: from 67% A (water, 0.1% trifluoroacetic acid) to 58% B (80% acetonitrile, 0.1% trifluoroacetic acid) in 60 min. Single fractions of 1 min were collected, monitoring the absorbance at 214 nm, and tested for bioactivity as described below. Fractions 13+14 contained the biologically active peptides according to this invention.

For the fifth purification step of PHC-1 and PHC-2 the obtained bioactive fractions 13+14 were further separated on an analytical RP-C18 column (5 μm, 30 nm, 1.0×25 cm; Vydac; flow rate: 1.8 ml/min) using the following gradient and buffers: from 70% A (water, 0.1% trifluoroacetic acid) to 45% B: (80% acetonitrile, 0.1% trifluoroacetic acid) in 45 min. Single fractions of 1 min were collected, monitoring the absorbance at 214 nm, and tested for bioactivity as described below. Fractions 19+20 contained the biologically active peptides according to this invention.

For the sixth purification step of PHC-1 and PHC-2 the obtained bioactive fractions 19+20 were loaded onto an analytical RP-C18 column (5 μm, 30 nm, 0.46×25 cm; YMC, Schembeck, Germany; flow rate: 0.6 ml/min) using the following gradient and buffers: from 77% A (water, 0.1% trifluoroacetic acid) to 38% B (80% acetonitrile, 0.1% trifluoroacetic acid) in 45 min. Single fractions of 1 min were collected, monitoring the absorbance at 214 nm, and tested for bioactivity as described below. Fraction 20 contained the biologically active peptides according to this invention.

For the seventh purification step of PHC-1 and PHC-2 the obtained bioactive fraction 20 was loaded onto an analytical RP-C18AQ column (3 μm, 0.1×25 cm; Reprosil-pur, Fa. Maisch, Ammerbuch 3, Germany; flow rate: 0.02 ml/min) using the following gradient and buffers: from 77% A (water, 0.1% trifluoroacetic acid) to 38% B (80% acetonitrile, 0.1% trifluoroacetic acid) in 45 min. Single fractions of 0.5 min were collected, monitoring the absorbance at 214 nm, and tested for bioactivity as described below. The purified, biologically active peptides PHC-1 and PHC-2 were found in the bioactive fractions 45 and 46 (FIG. 1B).

A fraction resulting from cation-exchange chromatography was subjected to reverse phase-HPLC (C 18 column) and the biological activity, of the fractions on human CCR5 was assayed (dashed line). Fifth and final purification step of the active material by reverse phase-HPLC (C4 column); The active peak (dashed line) was analyzed by mass spectrometry and Edman degradation, identifying PHC-1 (Mr 7795) and full-size HCC-1 (Mr 8673) as the major compounds in this fraction. Time-course of the CCR5 stimulatory activity generated by tryptic digestion of HCC-1. RP-HPLC chromatogram of trypsin-digested HCC-1 (1 h incubation), showing the generation of bioactive (shaded) PHC-1 (Mr 7795) as one of the main degradation products. Amino acid sequence of the N-terminal part of full-size HCC-1, PHC-1, HCC-3 and other chemokines active on CCR1, CCR3 or CCR5. The asterisks mark the first two conserved cysteines.

### Preparation of Additional Chemokine Compounds

The synthesis of additional N-terminally truncated chemokine compounds was performed using methods known to those of skill in the art. Briefly, the synthesis of additional chemokine compounds was carried out using TentaGel-R-Trt Asn(Trt) Fmoc resins (0.11-0.17 mmol/g) obtained from RAPP Polymere on a 433A peptide synthesizer (Perkin-Elmer/ABI). In general, the peptide chain assembly was performed with conductivity monitoring of Fmoc deprotection using HBTU/HOBt activation in NMP at a scale of 0.1 mmol. Fmoc-deprotection cycles were extended for 2 min. The following amino acids (residue numbers refer to the amino acid positions in SEQ ID NO: 4) showing incomplete acylation ratios were double-coupled: Asp66-Val64; Asn58; Thr49; Ile45-Ser38; Ile25-Lys24; His12 and Gly9. Ser41 was coupled four times. The second coupling step of Asn58; Thr49; Ser41-Ser38; Ile25-Lys24, and Gly9 was extended to two hours for Cys40 and His12 to one hour. Unreacted α-Amino-groups were regularly acetylated with a capping solution containing 10% acetic anhydride/5% DIPEA in NMP. After Pro43 the automated peptide synthesis was interrupted. Lys42 was double-coupled manually for 2 and 14 h using a symmetric anhydride activation procedure as described in detail previously (10 eq. amino acid; Atherton (1989) Solid phase peptide synthesis. IRL Press, Oxford, England, p. 76). A testdeprotection of a small amount of the resin and LCMS-analysis were performed to control the coupling efficiency of this coupling step.

### Preparation of a Chemokine Compound Analog

One aspect of the present invention relates to chemokine compounds which are modified at the N-terminus by coupling or substitution with a chemical group. Methods for modifying chemokine compounds in this manner are known to those of skill in the art (see for example, Gaertner et al., 1996, J. Biol. Chem. 271: 2591). For example, to generate NNY-HCC-1[10-74] (SEQ ID NO: 19), the chemokine compound HCC-1[10-74] was first generated as described above, and subsequently, nonanoic acid (20 eq.) was activated with HBTU/HObT and double-coupled manually for 1 and 3 h to the resin. The resulting polypeptide resin was cleaved, deprotected, precipitated and isolated by RP-HPLC according to a protocol recently described for CCL15 (Escher et al., 1999, J. Pept. Res. 54: 505).

### Disulfide-Bond Formation

The oxidative folding of chemokine compounds of the invention was carried out in the presence of the redoxsystem cysteine and cystine. For example, linear chemokine compounds were first dissolved in a minimal amount of water (1-2 ml) and then diluted in freshly degazed folding buffer (0.05-0.1 mg/ml) consisting of 0.1 M Na₂HPO4, 0.2 mM EDTA, 6 M GdnHCl, 0.2 M cysteine and 0.4 M cystine (pH 7.5). The solution was dialyzed twice for 24 h at 4° C. against the degazed GdnHCl-free folding buffer. NNY-HCC-1[10-74] was directly dissolved in the folding buffer prior to oxidation. The folding process was monitored by regular comparison of analytical C18-HPLC elution profiles. After completion, the folding mixture was acidified with TFA and folded peptides were isolated by one semipreparative HPLC-purification step. The lyophilized products were analyzed by ESMS and HPLC.

### Example 2

### Peptide Analytics

The biologically active peptides obtained by the seventh chromatographic step (example 1) were subjected to a structure determination. Mass determination of the purified peptides was carried out on a Sciex API III quadrupol mass spectrometer (Sciex, Perkin-Elmer) with an electrospray interface (ESI-MS). The molecular mass of the newly identified peptide PHC-1 was determined to be 7795+/−0.9 Da. The molecular mass of the newly identified peptide PHC-2 was determined to be 7479+/−1 Da (FIG. 1E).

The newly identified biologically active peptides were sequenced on an 473 A gas-phase sequencer (Applied Biosystems) by Edman degradation with on-line detection of phenylthiohydatoin-amino acids using the standard protocol recommended by the manufacturer.

The following N-terminal sequence was obtained for PHC-1:

(SEQ ID NO: 5)

GPYHPSEXXFTYTTYKIPRQRIMDYYETNSQ . . .

X: no amino acid signal detectable

The following N-terminal sequence was obtained for PHC-2:

| | |
|---|---|
| HPSEXXFTYTTYKIPRQRIMDYYETNSQ. | (SEQ ID NO: 6) |

X: no amino acid signal detectable

A data bank comparison was performed on Swiss-Prot and EMBL and databases. A sequence homology was established and showed sequence identity of both PHC-1 and PHC-2 to the precursor sequence of human chemokine HCC-1 (accession No. Q16627) or human chemokine HCC-3 (accession No. Q13954). PHC-1 and PHC-2 could represent the naturally processed, biologically active forms of human chemokine HCC-1 and/or human chemokine HCC-3.

The molecular mass, of PHC-1 (7795 Da) was exactly in accordance with the theoretical mass of a peptide comprising the C-terminal amino acid residues 9-74 of the precursor sequence of human chemokine HCC-1 calculated to be 7795 Da. The molecular mass of PHC-2 (7479 Da) was exactly in accordance with the theoretical mass of a peptide comprising the C-terminal amino acid residues 12-74 of the precursor sequence of human chemokine HCC-1 calculated to be 7479 Da.

The processed chemokine PHC-1 was a strong competitor of ¹²⁵I-Rantes binding to CCR1 with a Ki of +0.023±0.007 mM (Ki±SEM). PHC-1 was a strong competitor of ¹²⁵I-Eotaxin binding to CCR3 with a Ki of 2.7±0.8 nM (Ki±SEM). PHC-1 was a strong competitor of ¹²⁵I-MIP 1-alpha binding to CCR5 with a Ki of 0.04±0.01 nM (Ki±SEM).

### Example 3

### Determination of Biological Activity

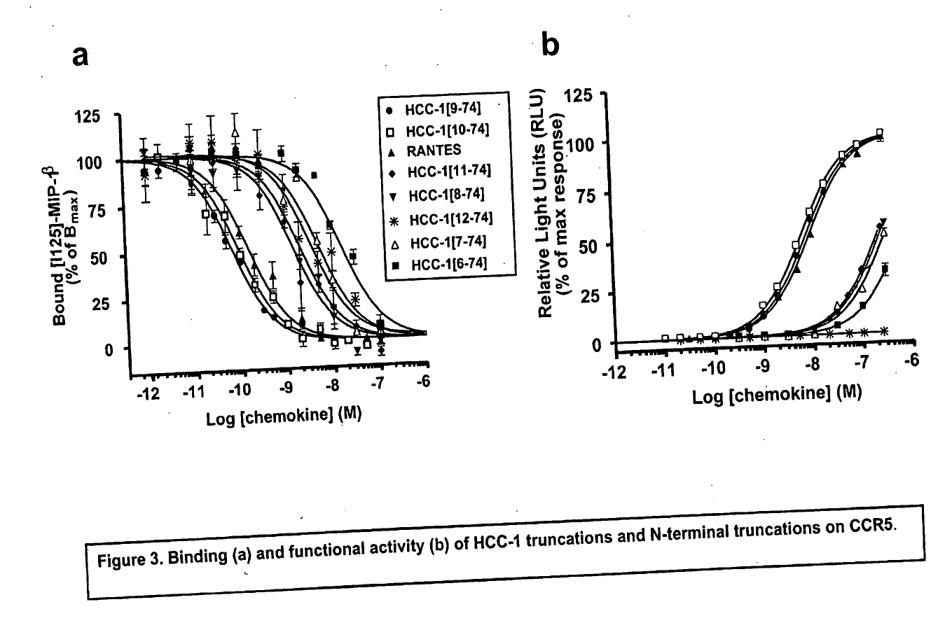

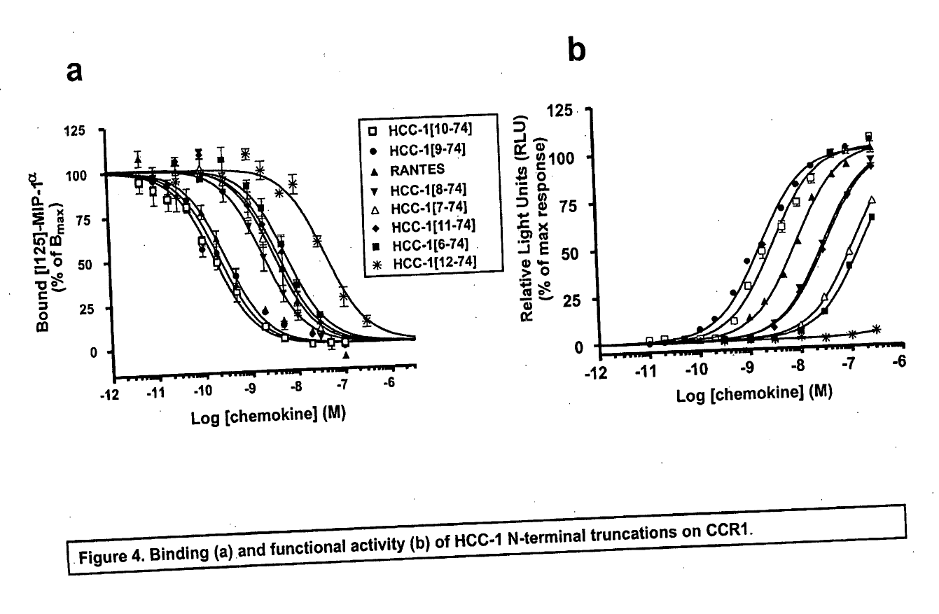

A biological assay was used measuring the activation of the chemokine receptor CCR5 by chemokine compounds useful in the present invention. Therefore, each of the fractions generated during the chromatographic purification procedure as described above (for PHC-1 and PHC-2), or each of the synthesized chemokine compound preparations was tested on CHO-K1 cells which were stably transfected with the chemokine receptor 5 (CCR5; AC P51681) or chemokine receptor 1 (CCR1; AC P56482). Activation of the cells via the CCR5 or CCR1 receptors was measured by the following Aequorin assay: Measurement of intracellular calcium increases was performed as described (Stables J et al 1997, Anal, Biochem. 252, 115-126). The various CHO-K1 cells stably expressing CCR5 or CCR1, mitochondrial apoaequorin and Gα16 were collected from plates with Ca²/Mg²⁺-free phosphate buffer saline (Life Technologies, Gaithersburg, Md.) supplemented with 5 mM EDTA, pelleted for 2 min at 1000×g and resuspended in D-MEM-F12 (Life Technologies, Bethesda) at a density of 5×10⁶ cells/ml. The active aequorin was reconstituted by incubation of cells during 4 hours at room temperature with 5 μM coelenterazine h (Molecular Probes, Eugene, Oreg.) in D-MEM-F12 medium containing 0.1 mg/ml bovine serum albumin. Following this incubation, cells were diluted tenfold in the same buffer and stirred for 30 minutes before measurement. 50 μl of the cell suspension was then injected onto 96-well plates containing the samples to be tested. The integrated light emission was recorded over 30 s with a Microbeta Jet (Wallac) or a Microlumat luminometer (EG&G Berthold). The final results were plotted as a percentage of the response obtained with 1 μM ATP. The purified peptides PHC-1 (corresponding to the sequence of the peptide HCC-1, residues 9-74) and HCC-1[10-74] (SEQ ID NO: 17) showed potent activation of both the CCR5 and CCR1 transfected cells. In comparison, the known beta-chemokines Rantes and NIP 1-alpha were used as positive controls. Chemokine compounds HCC-1[11-74], HCC-1 [8-74], and HCC-1[7-74] also activated both the CCR5 and CCR1 receptors, but with less potency than PHC-1 and HCC-1 [10-74]. EC₅₀ values for each of the chemokine compounds of the present invention are presented in FIG. 4. In comparison, the peptides HCC-1, residues 1-74 and HCC-1, residues 6-74 showed either a far less potent effect or no effect at all on the CCR5 and CCR1 transfected cells. Data is presented in FIGS. 3 and 4. Results are expressed as Relative Light Units (RLU). Symbols represent the same chemokines as for binding panels. Binding and functional assay results are representative of at least 3 independent experiments.

### Example 4

### Binding of PHC-1 to Different Chemokine Receptors (Binding and Aeguorin Assays)

The chemokine compounds of the invention bound with affinity binding to diverse chemokine receptors.

For determination of its binding properties, CHO-K1 cells were used which were stably transfected with the chemokine receptors CCR1, CCR3, CCR5. Therefore, crude membrane extracts prepared from the CHO-K1 cell lines expressing the various GPC were used in radioligand binding assays. Competition binding assays were performed in Minisorp tubes (Nunc, Roskilde, Denmark) in a total volume of 100 μl containing 0.1 nM iodinated ligand as tracer, variable concentrations of competitors and defined amount of membranes. Total binding was measured in the absence of competitor and non-specific binding was measured with a 100-fold excess, of unlabelled ligand. Samples were incubated for 90 minutes at 25° C. then filtered through GF/B filters presoaked in 0.3% polyethylenenimine. Filters were counted in a gamma scintillation counter (Packard). Binding parameters were determined with the PRISM software (Graphpad software, San Diego, Calif.) using non-linear regression applied to a one site competition model.

The processed chemokine PHC-1 was a strong competitor of ¹²⁵I-Rantes binding to CCR1 with half-maximal inhibitory concentration of 2.3±0.7 nM. (Ki±SEM). PHC-1 was a strong competitor of ¹²⁵I-Eotaxin binding to CCR3 with half-maximal inhibitory concentration of 78±14 nM (Ki±SEM). PHC-1 was a strong competitor of ¹²⁵I-MIP 1-alpha, binding to CCR5 with half-maximal inhibitory concentration of 0.04±0.01 nM (Ki±SEM).

The activity of PHC-1 was tested on CCR1 and CCR3. PHC-1 was more active than RANTES on CCR1 (EC₅₀±SEM: 2.8±0.8 nM compared to 6.3±1.1 nM) and a reasonably good agonist for CCR3 (EC₅₀: 78±14 nM)), while it was inactive on CCR2, CCR4, CCR6, CCR7, CCR8, CCR9, CXCR1 and CX3CR1.

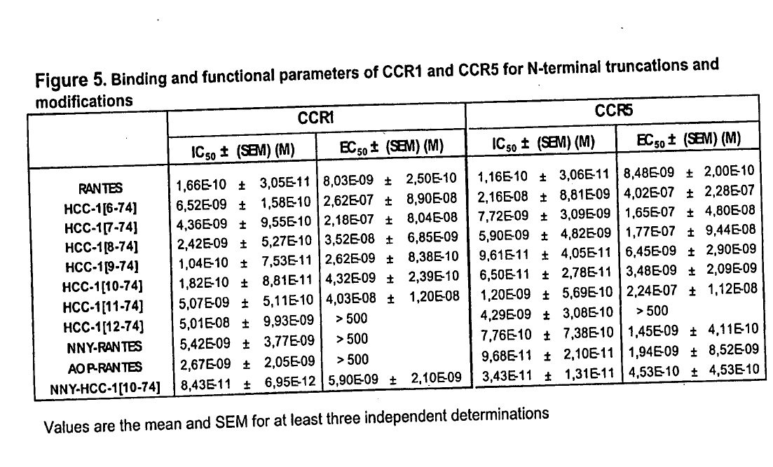

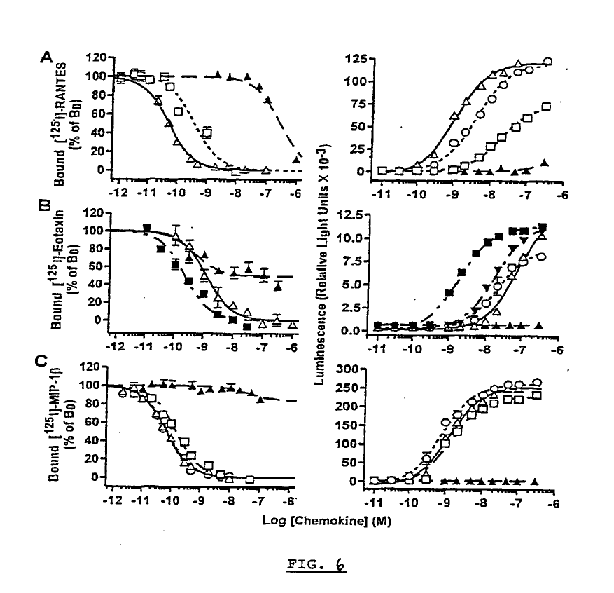

FIG. 6 represents the compound PHC-1, full size HCC-1 and reference chemokines tested on CCR1, CCR3 and CCR5. Competition binding assays were performed with [¹²⁵I]-RANTES for CCR1, [¹²⁵I]-Eotaxin for CCR3 and [¹²⁵I]-MIP-1, for CCR5. PHC-1, HCC1, MIP-1, RANTES, Eotaxin and MCP-4 were used as competitors. FIGS. 3 and 4 show the binding of additional chemokine compounds of the invention to the CCR5 and CCR1 respectively. Competition assays were performed with [¹²⁵I]-MIP-β1 for CCR5, and [¹²⁵I]-MIP-α1 for CCR1. Each of the chemokine compounds tested was capable of binding both the CCR5 and CCR1 receptors. IC₅₀ values for the competitive binding data shown in FIGS. 3 and 4 are shown in FIG. 5.

The results were normalized for binding in the absence of competitor (100%) and non-specific binding (0%). Functional aequorin-based assays (right panels) were run using CHO-K1 cells expressing the chemokine receptor, apoaequorin and C.

### Example 5

### Binding and Activation of Chemokine Compound Analogs on CCR5 and CCR1

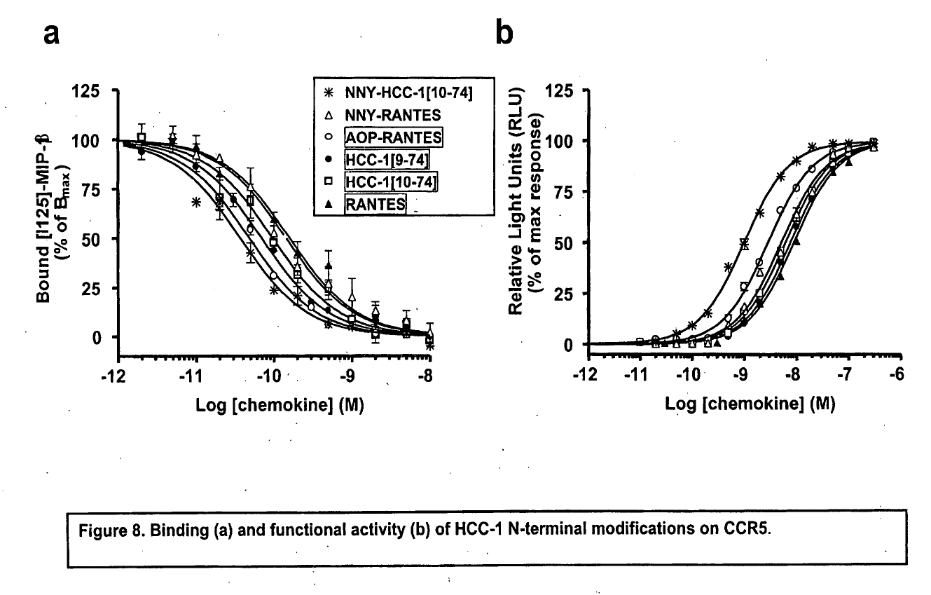

The chemokine compound analog NNY-HCC-1 [10-74] was tested for its ability to bind to and activate the CCR5 and CCR1 chemokine receptors. Binding and activation assays were carried out as described above in Examples 3 and 4. NNY-HCC-1 [10-74] was a potent and efficacious activator of both CCR5 and CCR1. In addition, NNY-HCC-1[10-74] was able to bind both receptors at a concentration similar to that of the non-analog form of HCC-1[10-74]. Binding and activation data for the CCR5 and CCR1 receptors are shown in FIGS. 7 and 8, respectively.

### Example 6

### Biological Activity of PHC-1 on Natural Cell Populations for its Calcium Mobilization and Chemotactic Properties

The migration of cells was assessed by a 48-well microchemotaxis chamber technique (Neuroprobe, Gaithersburg Md.). The lower compartment of the chamber was loaded with aliquots of 0.1% BSA medium or of each of the different chemokine concentrations (diluted in 0.1% BSA medium). The upper compartment of the chamber was loaded with a 55-μl cell suspension (5×10⁵ cells/ml in BSA medium) of cells which were previously isolated and washed three times in BSA medium. The two compartments were separated by a polycarbonate PVPF filter, 5 μm or 8 μm pore size according to the type of cell tested, coated with 20 μg/ml human collagen type IV for 2 h at 37° C. The chamber was incubated for 30 to 180 minutes at 37° C. in humidified air with 5% CO₂. At the end of the incubation period, the filter was removed, fixed, and stained with a Diff-Quik kit.

For each chemokine concentration tested, cells migrating through to the underside of the filter were counted in three high power fields (×500) by light microscopy (after coding the samples) in triplicate. Since the results of several experiments were combined in order to evaluate the migratory response, and since variation in potency of migration was observed between different experiments, the response to each of the chemokine concentrations is shown as a chemotaxis index. The chemotaxis index in each experiment was evaluated by calculating the following ratio: chemotaxis index=the mean of the number of cells migrating to a specific chemokine concentration/the mean of the number of cells migrating to BSA medium (=O ng/ml chemokine).

The data, are presented as the mean and standard errors (S.E.) of chemotaxis indices of 3-4 experiments. The baseline level of the number of transfected cells migrating was in a similar range for all the cells tested (1-5), with a mean of 6.7, ranging from 2-18 cells per high power field. Due to the large size of the cells and to limitations at the size of the high power field, the maximal response was limited to 70-80 cells per high power field. The p values of migration in response to each of the chemokine concentrations in comparison to migration in response to BSA medium were calculated based on the actual numbers of migrating cells using a Student's t test.

For intracellular calcium measurements, the cells were loaded for 30 min at room temperature with Fura-2AM. Calcium transients were monitored by an LS 50B spectrofluorimeter (Perkin Elmer) as described in C. Grynkiewicz, N. Poenie, R. Y. Tsien, J. Biol. Chem. 260, 3440 (1985).

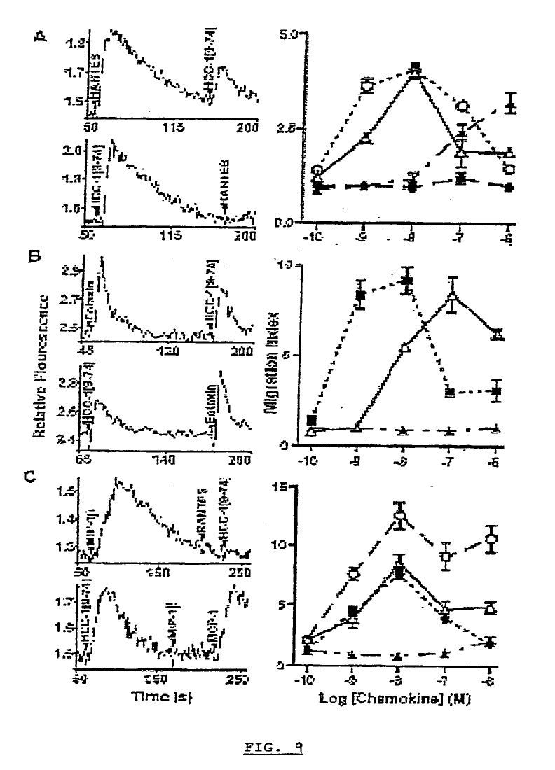

In monocytes, PHC-1 induced robust calcium fluxes, comparable to those evoked by RANTES (FIG. 9A). 50 nM PHC-1 totally desensitized these cells to further stimulation by the same ligand (data not shown), and abrogated further response to 50 nM RANTES as well, while PHC-1 remained capable of mobilizing calcium at reduced levels after a first stimulation by RANTES (FIG. 9A). Prior stimulation by full size HCC-1, MIP-1β or higher RANTES concentrations could not completely abolish the response to PHC-1. Similar results were obtained with the monocytic cell line THP-1. These results suggest that CCR1 and CCR5 mediate part of the functional response of monocytes to PHC-1. In chemotaxis assays with monocytes, PHC-1 was as potent (maximal migration observed at 10 nM) and efficient as RANTES. It was 100-fold more potent than full size HCC1, which appeared as a weak but efficient chemoattractant (C. L. Tsou et al. (1998) J. Exp. Med. 188, 603). PHC-1 mobilized calcium less efficiently than eotaxin in eosinophils (FIG. 9B). The eotaxin response was slightly reduced following prior exposure to PHC-1, while the activity of PHC-1 was unaltered following eotaxin stimulation (FIG. 9B) and, strongly inhibited after MIP-1α stimulation. These results substantiate the role of PHC-1 as a weak agonist of CCR3, and support the involvement of CCR1 in the functional response of eosinophils to PHC-1. In chemotaxis assays, PHC-1 was less potent but almost as efficacious as eotaxin on eosinophils from most donors. For one of the donors however, HCC-1 was weakly chemotactic at high concentrations only, which was attributed to the low CCR1 expression observed on eosinophils from some individuals (I. Sabroe et al. (1999) J Immunol. 162, 2946). PHC-1 but not full size HCC-1 mobilized calcium in interleukin-2 conditioned lymphoblasts. Complete cross-desensitization was observed between the responses to PHC-1 and MIP-1β, indicating that CCR5 is the principal receptor used by PHC-1 in these cells. Migration of lymphoblasts was stimulated by PHC-1 and NIP-1α but not by full size HCC-1 (FIG. 9C). PHC-1 also induced a small calcium flux in neutrophils and desensitization of this response by RANTES but not MIP-1 involved CCR1. No chemotaxis of neutrophils was observed with full length HCC-1 or PHC-1.

As shown in FIG. 9, monocytes, eosinophils and IL-2 stimulated lymphoblasts were tested for their functional response to PHC-1, full-size HCC-1 and reference chemokines. Stimulation of calcium mobilization and cross-desensitization experiments presented in FIG. 3 were performed with 50 nM chemokine concentrations. The cells were loaded with Fura-2AM and, the intracellular calcium concentration ([Ca⁺⁺]ᵢ) was monitored by ratiometric fluorescence measurement (R_{340/380}). Chemotaxis assays were performed in 48-well chambers using polycarbonate filter membranes. Cell migration in response to PHC-1, HCC-1, RANTES, Eotaxin or MIP-1 is reported as a migration index.

### Example 6

### Inhibition of HIV-1 Entry/Replication in Human Cells by PHC-1

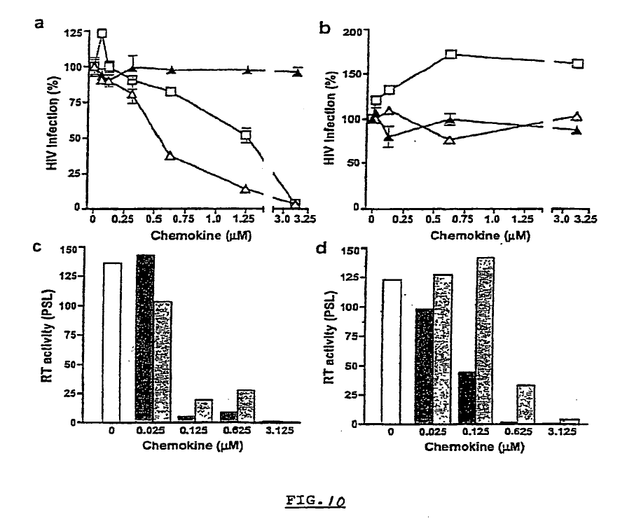

The processed chemokine PHC-1 is a potent inhibitor of HIV replication. The peptide bound to cofactors required for HIV-1 entry and efficiently blocked viral replication in cell culture. The anti-viral activity of PHC-1 was investigated in an infection inhibition assay. P4-CCR5 cells (NIH AIDS Research and Reference Reagent program) (P. Charneau et al. (1994) J. Mol. Biol. 241, 651) were grown in DMEM, supplemented with 10% FCS and 1 μg/ml puromycin. Cells were seeded in flat-bottom 96-well dishes, cultured overnight, and incubated with the chemokines for 2 hours prior to infection with virus containing 20 ng p24 antigen in a total volume of 50 μl medium. After overnight incubation, cells were washed twice and cultivated in fresh DMEM without chemokines. Three days after infection, the cells were lysed and β-galactosidase activity was measured. PBMC were isolated using lymphocyte separation medium (Organon Teknika Corporation). Cells were cultured in RPMI 1640 medium with 20% FCS and 50 U/ml IL-2, and virus production was measured by a reverse transcriptase assay as described (B. J. Potts, in Techniques in HIV Research, A. Aldovini, B. D. Walker, Eds. (Stockton, N.Y., 1990), pp 103-106). Both HCC-1[9-74] and RANTES inhibited infection by the macrophage-tropic strain YU2 (FIG. 10a). RANTES reduced YU2 infection of P4-CCR5 cells by more than 95% at 3.2 μM and by 50% at 1.3 μM. HCC-1[9-74] was slightly more efficient, blocking YU2 infection by 50% at 0.5 μM. Similar results were obtained using the macro-phagetropic strain JR-CSF. No inhibitory effect on YU2 infection was observed with full-length HCC-1. None of the three chemokines inhibited the T-tropic strain NL4-3 (FIG. 10b). In agreement with previously published results, RANTES enhanced infectivity of NL4-3 by about 50% at concentrations over 0.6 μM. In contrast, no enhancing effect on infectivity was observed with HCC-1 [9-74] (FIG. 4b). We also tested whether HCC-1[9-74] could inhibit HIV-1 replication in human peripheral blood mononuclear cells (PBMC). As shown in FIGS. 10c & d, replication of the macrophage-tropic strain JR-CSF was blocked significantly by HCC-1[9-74] and RANTES at concentrations of 125 nM, while concentrations of 625 nM were necessary for the YU2 strain. No inhibition of NL4-3 replication was observed, and HCC-1 was ineffective for all strains.

As shown in the FIG. 10, cells expressing both CCR5 and CXCR4 co-receptors were infected with the YU2 strain which uses CCR5, and the NL4-3 strain, which uses CXCR4, in the presence of HCC-1, PHC-1 or RANTES. The data represent the mean and s.e.m. for points performed in triplicate. C, D. Human PBMC were infected with YU2 and JR-CSF in the presence of HCC-1, PHC-1, or RANTES. The data represent the reverse transcriptase activity measured in culture cell supernatants harvested 14 days after infection. Results are expressed as photo-stimulated-luminescence (PSL) units.

### Example 7

### Generation, of Biologically Active PHC-1 by Tryptic Digestion of Human Chemokine HCC-1, Amino Acid Residues 1-74

The amino acid sequence of both newly identified biologically active peptides corresponds to the C-terminal sequence of the human chemokine HCC-1, which originally comprises 74 amino acid residues and was formerly described to occur in nanomolar amounts in human plasma (Schulz-Knappe et al. 1996, Journ. Exp. Med. 183, 295-299). Since the 74 amino residue comprising chemokine HCC-1 with the molecular mass of 8673 Da was completely inactive on CCR5 receptor activation and binding and showed only marginal binding to the CCR1 receptor (Ki 100 nM), we used the 74 amino residue comprising human chemokine HCC-1 as a educt in a digestion assay with the enzyme trypsin to obtain sufficient amounts of the 65 residue peptide PHC-1 for biological testing. After one hour of incubation (enzyme/substrate-ratio (w/w) 1/100) the 74 residue peptide was in part cleaved into smaller fragments. One cleavage product, the newly identified biologically active peptide PHC-1, comprising residues 9-74 of the HCC-1 precursor sequence, with the molecular mass of 7795 Da was obtained. The peptide of this invention was further purified to homogeneity (purity>99%) by two steps of analytical reverse phase chromatography. The peptide PHC-1 obtained from the tryptic digestion of biologically inactive HCC-1, 1-74 was tested in the biological assays as described above and showed full biological activity (FIG. 6). In contrary, the peptides HCC-1, residues 1-74 and HCC-1, residues 6-74 showed no effect in the assays described.

### Example 8

### Identification of the Protease that Cleaves Full Size HCC-4 into PHC-1

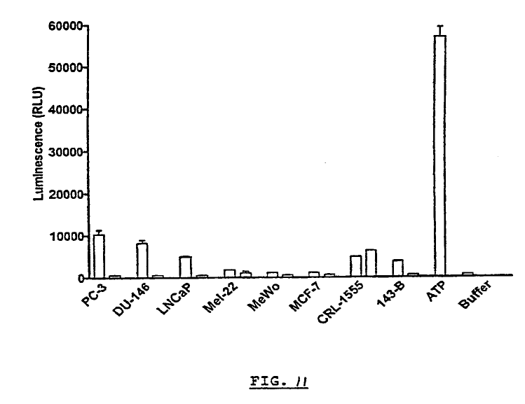

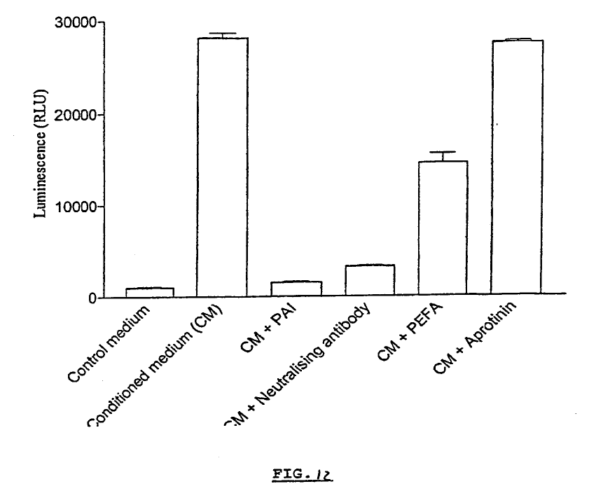

Several human cell lines were screened in order to identify a natural source for the protease generating PHC-1. 1 μM full size HCC-1 was added to the culture medium of 8 tumoral cell lines: PC-3 (prostatic carcinoma cell line, ATCC # CRL-1435, grown in Nutrient medium Ham's F12 with 10% foetal calf serum, 1 mM sodium pyruvate, 1 mM glutamate, 100 U/ml penicillin and 100 μg/ml streptomycin), Du-146 or 52, (adenocarcinoma cell line (ATCC HTB-81), grown in DMBM medium with 10% foetal calf serum, 1 mM sodium pyruvate, 1 mM glutamate, 100 U/ml penicillin and 100 μg/ml streptomycin), 143-B (osteosarcoma cell line, ATCC # CRL-8303, grown in DMEM medium with 10% foetal calf serum, 1 mM sodium pyruvate, 1 mM glutamate, 100 U/ml penicillin and 100 μg/ml streptomycin), MeI-22 and MeWo (melanoma cell lines, grown in DMEM medium with 10% foetal calf serum, 1 nM sodium pyruvate, 1. mM glutamate, 100 U/ml penicillin and 100 μg/ml streptomycin), MCF-7 (adenocarcinoma cell line, ATCC # HTB-22, grown in RPMI1640 medium with 10% foetal calf serum, 1 mM sodium pyruvate, 1 mM glutamate, 100 U/ml penicillin and 100 μg/ml streptomycin) and CRL-1555 (epidermoid carcinoma cell line, ATCC # A431, grown in RPMI1640 medium with 10% foetal calf serum, 1 mM sodium pyruvate, 1 mM glutamate, 100 U/ml penicillin and 100 μg/ml streptomycin). Following an incubation of 48 hours at 37° C., the culture medium was centrifuged and tested in an aequorin assay using CCR5 expressing cells, as described in example 3. FIG. 11 shows that PC-3, DU-146 and 143-B activated full size HCC-1. A non specific response was only obtained with the CRL-1555 culture medium without addition of full size HCC-1. Serum-free conditioned media obtained from these different cell lines were also able to activate full size HCC-1, indicating that the protease generating PHC-1 was soluble. As PC-3 secretes high amount of urokinase, a serine protease, several serine protease inhibitors were tested in PC-3 conditioned medium incubated with 500 nM full size HCC-1, for 6 hours at 37° C. As shown in FIG. 12, all the protease inhibitors decreased the activation of full size HCC-1. 3 μg/ml PAI-1, a specific urokinase inhibitor, was more efficient than 4 mM Pefabloc; 2 μg/ml aprotinin was inactive. Furthermore, a specific blocking urokinase antibody (monoclonal neutralizing antibody 394 against human uPA B-chain, American Diagnostica, USA) also inhibited the activation of full size HCC-1. Purified CHOAY urokinase (Sanofi Wintrop) was then incubated at 37° C. for 40 min with 500 nM full size HCC-1. The reaction product was purified on reverse phase-HPLC and the fractions were tested in the aequorin assay with CCR5 expressing cells. The active fractions were then analysed by mass spectometry and Edman degradation, identifying PHC-1 and full size HCC-1 as the major compounds in these fractions. As specified above, trypsin generated PHC-1 then degrades PHC-1 into inactive fragments when the incubation time is increased. 50 nM PHC-1 was incubated with 10 or 100 IU purified urokinase. No change of activity was observed after an incubation of 90 min at 37° C., indicating that urokinase do not degrade PHC-1.

What is claimed is:

1. An isolated chemokine compound comprising an amino acid sequence which is at least 95% homologous with SEQ ID NO: 1, wherein said compound does not comprise the amino acid sequence SEQ ID NO: 3, SEQ ID NO: 4 or their biologically active amidated, acetylated, phosphorylated and/or glycosylated derivatives and wherein the isolated chemokine compound activates the chemokine receptor CCR-3 and/or CCR-5 as measured in an aequorin assay.

2. The isolated chemokine compound of claim 1 wherein the amino acid sequence comprises SEQ ID NO: 1, SEQ ID NO: 2 or biologically active fragments or portions thereof.

3. The isolated chemokine compound of claim 1 wherein the amino acid sequence comprises SEQ ID NO: 1, SEQ ID NO: 2 or biologically active fragments or portions thereof, modified by or linked to one or more amide, acetyl, phosphoryl and/or glycosyl groups.

4. The chemokine compound of claim 2, wherein said compound is capable of binding to a chemokine receptor selected from the group consisting of CCR-1, CCR-3 and CCR-5.

5. The chemokine compound of claim 3, wherein said compound is capable of binding to a chemokine receptor selected from the group consisting of CCR-1, CCR-3 and CCR-5.

6. A pharmaceutical composition comprising the compound of claim 1 and an pharmaceutically acceptable carrier.

7. A pharmaceutical composition comprising the chemokine compound of claim 5 and a pharmaceutically acceptable carrier.

8. A pharmaceutical composition comprising the chemokine compound of claim 6 and a pharmaceutically acceptable carrier.
